(19) United States
(12) Patent Application Publication
(10) Pub. No.: US 2018/0042899 A1
(43) Pub. Date: Feb. 15, 2018

(54) Method of Treating Transplant Rejection and Autoimmune Diseases

(71) Applicant: Wisconsin Alumni Research Foundation, Madison, WI (US)

(72) Inventors: Joshua David Mezrich, Madison, WI (US); Christopher A. Bradfield, Madison, WI (US)

(21) Appl. No.: 15/723,826

(22) Filed: Oct. 3, 2017

Related U.S. Application Data
(63) Continuation of application No. 13/969,996, filed on Aug. 19, 2013, now abandoned.
(60) Provisional application No. 61/684,987, filed on Aug. 20, 2012.
(60) Provisional application No. 61/740,082, filed on Dec. 20, 2012.

(51) Int. Cl. A61K 31/4045; A61K 31/655; A61K 45/06; A61K 31/404

(52) U.S. Cl. CPC A61K 31/4045; A61K 45/06; A61K 31/404; A61K 31/655; A61K 2300/00

(57) ABSTRACT

A method of treating autoimmune diseases and transplant rejection, comprising the step of treating the autoimmune or transplant patient with an effective amount of SU-5416 is disclosed.

### CROSS-REFERENCE TO RELATED APPLICATION(S)

This application is a continuation of U.S. patent application Ser. No. 13/969,996 filed Aug. 19, 2013, which is claims the benefit of U.S. Provisional Patent Applications 61/684,987 filed Aug. 20, 2012, and 61/740,082, filed Dec. 20, 2012, which both are incorporated herein by reference in its entirety.

### STATEMENT REGARDING FEDERALLY SPONSORED RESEARCH OR DEVELOPMENT

This invention was made with government support under RR025012 and ES005703 awarded by the National Institutes of Health. The government has certain rights in the invention.

### BACKGROUND

Autoimmune diseases arise from an inappropriate immune response of the body against substances and tissues normally present in the body. The immune system mistakes “self” as foreign or a pathogen and destroys its own cells. Similarly, transplant rejection occurs when transplanted tissue is rejected and destroyed by the recipient's immune system. Transplant rejection can be lessened by determining the molecular similitude between donor and recipient and by use of immunosuppressant drugs after transplant.

Immune diseases and transplant rejection are treated by suppressing the immune system through treatment with drugs. This is a double-edged sword because the immune suppression protecting one from one's own immune response also results in suppression of response to pathogens. A more effective therapy would result if the immune system could be directed or manipulated to respond in an appropriate manner.

There is a need in the art for alternate methods of immune modulation for the treatment of autoimmunity and transplant rejection.

### BRIEF SUMMARY OF THE INVENTION

The present invention generally relates to a method of treating a patient with an autoimmune disease or inflammatory disorder. In one embodiment, the method comprises the step of treating a patient with a therapeutic composition comprising a sufficient amount of SU5416 such that symptoms of disease are alleviated.

In its first aspect, the present invention provides methods of treating an autoimmune patient includes administering a therapeutic composition comprising SU5416 on its own or as part of a composition with other therapeutic compounds. For example, SU5416 can be used together with Thymoglobulin (rATG), Campath, costimulatory blockade, infliximab, etanercept, adalimumab, golimumab, natalizumab, cytokines IL-10, IL-2, TGF-β, NSAIDs, corticosteroids, immune suppressants or TNF-α inhibitors.

In some cases, a therapeutic composition comprising SU5416 and a plurality of cytokines would be administered to an autoimmune patient. This plurality of cytokines can either include cytokines that drive T-cell differentiation into a more robust T-regulatory population or alter the direction of differentiation to a T-effector cell population, for treatment in cases of diseases like asthma, as part of the treatment of these autoimmune patients. Autoimmune diseases include, but are not limited to, rheumatoid arthritis, diabetes, asthma, Crohn's Disease, inflammatory bowel disease, psoriasis, interstitial fibrosis, systemic lupus erythematosus, uveitis, and glomerulonephritis. The therapeutic composition typically contains 30-150 mg/m² of SU5416.

In its second aspect, the invention provides methods of treating a transplant patient includes administering a therapeutic composition that comprises SU5416, SU5416 and other therapeutic compounds such as Thymoglobulin (rATG), Campath, costimulatory blockade, infliximab, etanercept, adalimumab, golimumab, natalizumab, cytokines IL-10, IL-2, TGF-β, NSAIDs, corticosteroids, immune suppressants and TNF-α inhibitors.

The autoimmune disease or inflammatory disorder can be treated with the present method include, but are not limited to, rheumatoid arthritis, psoriatic arthritis diabetes, multiple sclerosis, interstitial fibrosis, lupus, glomerulonephritis, Crohn's Disease, inflammatory bowel disease, psoriasis and autoimmune eye diseases (uveitis).

The transplant rejections are preferably the solid organ transplant rejections. Non-liming examples of the solid organ transplant include lung transplant, bronchiolitis-obliterans syndrome (BOS), heart transplant, kidney transplant, liver transplant, pancreas transplant, and corneal transplant.

In preferred embodiments, the treatment for an autoimmune patient or a transplant patient is applied when at least one symptom of the autoimmune diseases or the transplant rejection is diagnosed. More preferably, the symptom to be treated is at acute phase.

The dose of SU5416 to be administered for treating autoimmune diseases or transplant rejections is 30-150 mg/m². Preferably, the dose of SU5416 is 85-145 mg/m².

In its third aspect, the present invention relates to compostions for treating autoimmune disease or transplant rejection comprising (a) a effective amount of SU5416 and at least one additional therapeutic compound described above.

In its fourth aspect, the present invention also provides methods of discovering new therapeutic compounds, comprising the steps of (a) examining a target chemical for similar structure or function to SU5416, and (b) identifying a chemical with sufficient functional similarity to SU5416, wherein the treatment with the test chemical produces therapeutic results. The methods may additionally comprising the step of (c) treating an autoimmune or transplant patient with the identified chemical. By using the method, a suitable chemical equivalent of SU5416 can be discovered based on its activity of activating AHR and/or inhibiting VEGFR.

### BRIEF DESCRIPTION OF THE DRAWINGS

FIG. 1A-B is a set of graphs depicting a small molecule library screen for AHR agonists (FIG. 1A) and a screen for agonists of AHR^{d} (FIG. 1B). FIG. 1A is a collection of 4160 compounds was screened for the induction of the DRE-driven luciferase in the human hepatoma 101L cell line. In 384-well plates, 100 μL media containing 70% confluent 101L cells was incubated with 10 μM of each test compound (1% v/v DMSO) for 24 hours. Dotted line indicates 3-fold induction. FIG. 1B. is a screen for agonists of the AHR^{d}. The AHRd-15 cell line was treated with 1 μM of the 98 compounds identified from the primary screen, 2 nM TCDD or DMSO and EROD activity was determined. Dashed line indicates 5-fold induction.

FIG. 2A-C is a set of graphs that address agonism of AHR. FIG. 2A is bar graph of the induction of DRE-mediated AHR transcription by SU5416 and FIG. 2B is a bar graph of the induction of DRE-mediated transcription of ARNT by SU5416. FIG. 2C is a graph of competitive ligand binding to AHR. The AHR-mutant C35 cell line was transfected with the AHR^{b}, lacZ gene and a 3 xDRE-Luc construct. Controls were transfected with the empty pSPORT vector plus the reporter constructs. After 24 h, the cells were treated with 3 SU5416 or 0.3% (v/v) DMSO, then incubated for 18 more h. Induction of AHR activity was determined by normalizing the luciferase activity to β-galactosidase activity. White bars: Empty vector. Grey bars: AHR. Error bars: SD; (n=3). FIG. 2B. Induction of DRE-mediated transcription by SU5416 is ARNT dependent. The ARNT-deficient C4 cell line was transfected with the human ARNT or the pSPORT parent vector. These cells were also co-transfected, treated and assayed as in A. White bars: Empty vector. Grey Bars: ARNT. Error bars: SD; (n=3). FIG. 2C. SU5416 is a ligand of the AHR. The hepatic cytosolic fraction from C57BL/6J mice was incubated with 1 nM of the radioligand ¹²⁵BR2N3DpD, in the presence of increasing concentrations of competitor, SU5416, TCDD, BNF or 1,2-Benzanthracene. Ordinate: Specifically bound radioligand in the presence of competitor divided by specifically bound radioligand in the absence of competitor. Abscissa: The concentration of competing ligand, represented as log of molar concentration. Each data point represents the average of two determinations. Competitive binding to the C57BL/6J cytosol produced the IC₅₀ values of SU5416=2.1 nM, TCDD=1.5 nM, BNF=2.8 nM, and 1,2-Benzanthracene=13.7 nM.

FIG. 3A-D is a series of graphs that compare activity of AHR agonists. FIG. 3A is a graph of in vitro dose curves for increasing doses of TCDD in rat hepatoma cells lines bearing the murine AHR^{d}. FIG. 3B is a graph of in vitro dose curves for increasing doses of SU5416 in rat hepatoma cells lines bearing the murine AHR^{d}. FIG. 3C is a graph of in vitro dose curves for increasing doses of BNF in rat hepatoma cells lines bearing the murine AHR^{d}. FIG. 3D is a graph of EROD activity from hepatic microsomal proteins isolated from mice orally dosed with BNF or SU5416.

FIG. 4A-D is a series of graphs comparing CYPA1 transcription in response to AHR activation by different agonists. FIG. 4A is a graph comparing the CYPA1 mRNA in wild-type and AHR^{d} rodent splenocytes after treatment with TCDD. FIG. 4B is a graph comparing the CYPA1 mRNA in wild-type and AHR^{d} rodent splenocytes after treatment with SU5416. FIG. 4C is a graph comparing the response of wild-type and AHR^{d} response to stimulation by TCDD. FIG. 4D is a graph comparing the response of wild-type and AHR^{d} response to stimulation by SU5416.

FIG. 5A-G is a series of graphs that compare the transcription of mRNAs in response to titrated doses of SU5416 and AHR signaling. FIG. 5A is a graph of mRNA levels of CYP1A1 in response to titrating doses of SU5416. FIG. 5B is a graph of mRNA levels of CYP1B1 and IDO in response to titrating doses of SU5416. FIG. 5C is a graph of CYP1A1 expression in wild-type and AHR null mice after treatment with IFN-γ or SU5416. FIG. 5D is a graph of IDO expression in wild-type and AHR null mice after treatment with IFN-γ or SU5416. FIG. 5E is a graph of IDO1 mRNA expression in pDC/T-cell co-culture. FIG. 5F is a graph of FoxP3 mRNA expression in pDC/T-cell co-culture. FIG. 5G is a diagram of FoxP3 and CD59 expression on naive T cells in the presence of TGF-β and either DMSO or SU5416 as detected by flow cytometry. FIG. 5A. Spleens were harvested from mice and processed in the standard fashion. Cells were incubated for 4 hours in culture with titrating doses of SU5416 as indicated, and afterwards mRNA was measured for CYP1A1. FIG. 5B. SU5416 upregulates CYP1B1 and IDO. Same assay as A, but mRNA for CYP1B1 and IDO were measured. FIG C. Upregulation of CYP1A1 mRNA is dependent on the AHR. Splenocytes from C57BL/6J or AHR^{-/-} mice were exposed to media, IFN-γ 100 ng/ml, or SU5416 500 nM for 4 hours. mRNA was then harvested and assayed for CYP1A1. nd represents “not detected”. ***—p≦0.001. FIG. 5D. IDO upregulation by SU5416 is AHR-dependent. Same assay as in C, but IDO mRNA was assessed. *—p≦0.05. *—p≦0.01. FIG. 5E. SU5416 induces IDO in the pDC/T cell coculture to a greater extent than TCDD. pDC/T cell coculture was utilized as described previously [25]. Culture was performed for 5 days with SU5416 500 nM, TCDD 10 nM, FICZ 100 nM, or control, at which point mRNA was harvested and measured for IDO. *—p≦0.05. ***—p≦0.001. FIG. 5F. SU5416 induces FoxP3 in the pDC/T cell coculture to a greater extent than TCDD. Same assay as in E, but mRNA was assayed for FoxP3. *—p≦0.05. **—p≦0.01. FIG. 5G. SU5416 enhances FoxP3 expression and CD39 on naïve T cells in the presence of TGF-β. Naïve T cells were placed in culture with DMSO (1:4×10⁴ dilution), TGF-β (2 ng/ml), or TGF-β (2 ng/ml) and SU5416 (250 nM), harvested on day 3, and analyzed by flow cytometry. All of the above figures are representative of 3 independent experiments.

FIG. 6 is a table of In utero exposure to SU-5416 stimulates closure of DV.

FIG. 7A-B is a gel picture and a graph comparing AHR protein levels and activation. FIG. 7A is a western blot for AHR protein. FIG. 7B is a graph comparing AHR^{d} activation after treatment with BNF or TCDD. FIG. 7A. A Western blot was performed using the whole cell lysate of AHRd-15 cells. Lysate from the AHR null BP8 parental cell line, and the hepatic cytosolic fractions from C57BL/6J and DBA/2J mice were included as size controls. Proteins were resolved by electrophoresis on a 7.5% acrylamide gel, and then probed with the BEAR-3 anti-AHR antibody. FIG. 7B. AHR^{d}-15 is responsive to TCDD, but not BNF. Dose-response curves were generated by treating AHR^{d}-15 cells with nM doses of TCDD and μM doses of BNF for 36 hours. Activation of the AHR^{d} was determined by quantifying EROD activity from whole cell lysate.

FIG. 8A-C are a series of graphs that compare SU5416 antagonism. FIG. 8A is a graph comparing TCDD and SU5416 activation of AHR. FIG. 8B is a graph comparing the antagonism response of SU5416 and TCDD by CH223191. FIG. 8C is a graph comparing the antagonism response of SU5416 with and without antagonist. FIG. 8A. 0.6×10⁶ Cells from a mouse hepatoma cell line H1L6.1c3, stably carrying a dioxin-responsive element (DRE)-driven firefly luciferase reporter gene were seeded in each well of a six-well plate overnight and were then treated with SU5416 100 nM or TCDD 1 nM for 4 hours through 96 hours. DRE activity was assayed by a luminometer at the time points shown. Data is presented as a percent of 100 nM TCDD response at those time points. FIGS. 8B-C. Characterization of antagonism of response of SU5416 by CH223191. FIG. 8B. SU5416 100 nM or TCDD 1 nM were tested with DRE-driven luciferase reporter cells with titrating doses of the antagonist for 4 hours as delineated in the figure. Data is presented as % response without inhibitor. FIG. 8C. SU5416 was titrated in culture with and without the antagonist (10 μM) for 4 hours. Results are presented as % maximal TCDD response at 100 nM.

FIG. 9A-D is a series of graphs that compare agonism of SU5416 and TCDD. FIG. 9A is a graph comparing CYP1A1 mRNA in splenocytes of wild-type and AHR^{d} mice following exposure to TCDD. FIG. 9B is a graph comparing CYP1A1 mRNA in splenocytes of wild-type and AHR^{d} mice following exposure to SU5416. FIG. 9C is a graph comparing DRE-mediated luciferase expression in cells transfected with AHR containing a valine point mutation or wild-type AHR after exposure to TCDD. FIG. 9D is a graph comparing DRE-mediated luciferase expression in cells transfected with AHR containing a valine point mutation or wild-type AHR after exposure to SU5416. Similar to FIG. 4, splenocytes from wild-type and AHR^{d} mice analyzed by qPCR for CYP1A1. Spleens from these mice were harvested and suspended in culture media, and exposed to titrating doses of A) TCDD B) SU5416. After 4 hours they were analyzed by qPCR for CYP1A1 analysis. The curves represent fold change and show the similar potency of these ligands. Each graph is representative of 3 independent experiments. FIG. 9C-D. Cells transfected with AHR containing a valine point-mutation show similar ED₅₀ to Cos-1 cells with AHR^{b} isoform. Cos-1 cells were transfected with an AHR containing the same point mutation (valine for alanine) thought to be responsible for the low affinity of the AHR^{d} isoform compared to AHR^{b}, and compared to the wild-type AHR response. These cells also harbor a luciferase gene next to the DRE. FIG. 9C. Cos-1 cells were exposed to TCDD. FIG. 9D. Cos-1 cells were exposed to SU5416. The graphs represent true luciferase values. They are representative of 2 independent experiments.

FIG. 10 is a diagram of IL-17 expression in naïve T-cells cultured in Th17 conditions then exposed to SU5416. SU5416 causes a small amount of IL-17 secretion at low doses. Naive T-cells were placed in Th17 conditions in culture (TGF-β4 ng/ml, IL-6 20 ng/ml) and exposed to titrating doses of SU5416 as indicated. After 3 days of culture, supernatant was harvested and tested for IL-17 by ELISA.

FIG. 11 is a diagram of skin graft survival after treatment with SU5416.

FIG. 12 is a chart showing the scoring of the skin grafts.

FIG. 13 is a table showing activation of the AHR by VEGF-2 kinase inhibitors, highlighting that not all VEGF inhibitors have the ability to bind to the AHR.

### DETAILED DESCRIPTION OF THE INVENTION

### Agonists of AHR

Agonists of the aryl hydrocarbon receptor (AHR) have been of interest to the pharmaceutical industry for many years. This interest originally stemmed from the observation that the AHR is a ligand-activated transcription factor that regulates the adaptive metabolism of xenobiotics [1] and because receptor binding is a known step in the carcinogenic and toxic action of environmental pollutants like 2,3,7,8-tetrachlorodibenzo-p-dioxin (TCDD) [2]. Thus, agonist of the AHR has commonly been considered a signature for drugs that up-regulate phase-I and phase-II metabolic systems and also for chemicals with pharmacological similarity to a known human carcinogen. As a result, AHR agonist has largely been considered a hazard signature for environmental chemicals and drugs in the pharmaceutical pipeline.

Recent insights related to the normal physiological role of the AHR are changing the accepted view of receptor agonist to one where agonist might be considered to hold therapeutic value. A number of recent reports are identifying new biological processes that might be influenced by endogenous receptor ligands. For example, descriptions of mice harboring a null allele at the Ahr locus indicate that receptor signaling plays an important role in normal cardiovascular development and function [3,4]. The therapeutic potential related to this biology is demonstrated by the observation that potent AHR agonists like TCDD can correct developmental aberrations in hepatic blood flow under conditions of AHR hypomorphism [5].

More recently, a role for the AHR in immunology has been suggested by reports that activation of this receptor with ligands, such as TCDD, can lead to the generation of regulatory T-cells (Tregs) [6], while activation with other ligands, such as formylindolo[3,2-b]carbazole (FICZ) can lead to Th17 cell formation [7]. The potential clinical importance of this finding is supported by the observation that TCDD is able to ameliorate the symptoms of experimental autoimmune encephalomyelitis (EAE) in mice, whereas FICZ aggravates this syndrome. Additional studies have supported the idea that ligands can play a role in improving allograft acceptance after transplantation [8]. The importance of the AHR in immunology has also been extended by a series of papers demonstrating the central importance of this receptor in the presence and maintenance of intraepithelial lymphocytes and lymphoid tissue inducer cells in the gut, highlighting that the AHR and its ligands play a role in normal physiology of the immune system and response to the outside environment [9,10,11].

### SU5416

SU5416, Semaxanib, is a potent selective inhibitor of receptor tyrosine kinases and has been evaluated in phase I, II and III clinical trials for the treatment of human cancer.

The chemical name for SU5416 is 3-[(2,4-dimethylpyrrol-5-yl)methylidenyl]-indolin-2-one and the compound is typically prepared from commercially available 3,5-dimethylpyrrol-2-carboxaldehyde by aldol condensation with indolin-2-one in ethanol in the presence of piperdine. (see Fong T A et al, Cancer res. 1999 Jan. 1; 59(1) 99-106.) SU5416 can also be purchased from a number of commercial chemical suppliers, such as Sigma-Aldrich.

SU5416 has been shown to inhibit vascular endothelial growth factor receptor 2 (VEGFR2) and research has suggested that the pharmacological inhibition of the activity of VEGFR2 represented a good strategy for limiting the growth of a wide variety of tumor types and combating cancer [19]. However, the phase III clinical trials showed poor results.

Suprisedly, we found that SU5416 is also an aryl hydrocarbon receptor (AHR) agonist with unique properties. An AHR agonist is considered to represent a poor characteristic for an anticancer drug because it promotes regulatory T-cells which can inhibit clearance of cancer cells. Activation of the AHR can also lead to up-regulation of xenobiotic metabolizing enzymes that might influence the half-lives of co-administered chemotherapeutic agents.

Specifically, we found that SU5416 favors induction of indoleamine 2,3 dioxygenase (IDO) in immunologically relevant populations such as dendritic cells in an AHR-dependent manner, leading to generation of regulatory T-cells in vitro. SU5416 activates the human AHR with a potency approaching 2,3,7,8-tetrachlorodibenzo-p-dioxin (TCDD) and also activates polymorphic murine receptor isoforms (encoded by the Ahr^{d} and Ahr^{b1} alleles) with similar potency.

These characteristics of SU5416 lead us to assert that SU5416 is an ideal clinical agent for treatment of autoimmune diseases and prevention of transplant rejection, two areas where regulatory ligands of the AHR have shown promise. Also, this novel discovery has significant implications on how SU5416 should be used for treatment of rejection or autoimmunity that would not be supported by its function as a VEGFR2 inhibitor alone.

There are three types of therapy for transplant rejections: (1) induction therapy that is used for a short period of time at the time of transplant, (2) maintenance therapy that is used chronically after transplant, and (3) acute therapy that is periodically used in an acute phase of an infection, such as acute rejection episodes. If the mechanism of SU5416 were purely related to VEGF inhibition as previously believed, one would wait a short period of time after the transplanted graft is vascularized, and then start SU5416 treatment in the recipient shortly thereafter as a maintenance therapy. This is because, based on this mechanism, the SU5416 treatment can block new blood vessel growth (angiogenesis) that occurs over time, leading to chronic infiltration of immune cells and other mediators of inflammation that can lead to rejection.

However, based on the new discovery that SU5416 is also an immunomodulator that can enhance T cell differentiation to regulatory T cells that reduce inflammation, we envision that SU5416 should be more responsive to acute and chronic rejection episodes to reduce inflammation (rejection) when it is diagnosed. The SU5416 treatment could be discontinued once biopsies or clinical indicators imply that rejection has been adequately treated. Typically, this type of acute therapy would last for a few weeks to months around the time of rejection, as opposed to chronic maintenance treatment over years, even the life of the patient.

This same logic applies to treatment with autoimmune flare-ups. If SU5416 were only a VEGFR2 inhibitor, then patients with autoimmunity would need chronic treatment over years to prevent the chronic angiogenesis that favors inflammation. However, as an immune modulator, SU5416 would be preferably used when clinical symptoms of autoimmunity are recognized and the treatment of SU5416 could be stopped once those symptoms resolve.

A second important distinction that this new discovery separates from the previous understanding of SU5416 is which drugs might be utilized with SU5416 in a combination therapy.

Typically, patients with transplant rejection or flare-ups of autoimmunity are treated with multiple drugs. As a ligand of the AHR, SU5416 has at least two distinguishable applications in combination therapy. The first is that as an immunomodulatory agent that enhances regulatory T cell generation and reduces Th17 cell differentiation, SU5416 can be combined with agents that can also enhance immunomodulation. One example of the suitable agents is costimulatory blockade (i.e. Belatacept®), which blocks secondary signaling of T cells and also enhances regulatory T cells.

However, if the mechanism of SU5416, as previously believed, were solely to function through VEGF inhibition, there would be less mechanistic support to consider combination therapy, since blocking angiogenesis would make other immunomodulatory drugs less effective and less able to be delivered to the site of inflammation.

In addition, as a ligand of the AHR, SU5416 can upregulate the Cytochrome P450 enzymes as all AHR ligands do and lead to metabolism of other drugs. Therefore, it will be useful to consider the clearance of any drugs used in combination with SU5416 and make appropriate adjustment in doses and measure for levels and efficacy.

Autoimmune Disease

The treatment of autoimmune diseases is typically with immunosuppressive medication, which decreases the immune response and, thus, dampens the destruction of “self” which is the basis for these diseases. Current immunosuppressants used in the treatment of autoimmune disease would typically include: corticosteroids, cytostatics, antibodies, drugs acting on immunophilins and TNF-α inhibitors. Corticosteroids such as glucocorticoids inhibit humoral immunity; cytostatics inhibit cell division and affect B and T-cells most; antibodies inhibit T-cells and cause their lysis, which inhibits cell-mediated immune reactions. These treatments all impair T-cells and their functioning as a way to eliminate the attack and destruction of self by the immune response. In addition to the immunosuppressive drugs, autoimmune patients are also treated with non-steroidal anti-inflammatory drugs (NSAIDs) and corticosteroids to mitigate inflammation and pain.

SU5416 has an immune modulatory function. Treatment with SU5416 either alone or in combination with other drugs would direct the differentiation of T-cells to regulatory T cells and produce an immune response that would control or countermand the attack of “self”. Regulatory T cells (T_{reg}) are a subpopulation of T cells which modulate the immune system, maintain tolerance to self-antigens, and abrogate autoimmune disease.

In one embodiment of the present invention, SU5416 is used for treating autoimmune diseases, including inflammatory disorders, comprising the step of treating the autoimmune patient with an effective amount of SU5416.

The autoimmune diseases and inflammatory disorders that may be treated by SU5416 include, but are not limited to, arthritis (including rheumatoid and psoriatic), diabetes, interstitial fibrosis, autoimmune eye disease (uveitis), lupus, Crohn's Disease, inflammatory bowel disease, psoriasis and kidney disease that is auto-immune in nature, such as glomerulonephritis.

Technically, there is no cure for autoimmune disease. Thus, one of the primary goals of the present treatment is to reduce or alleviate symptoms and control the immune response in patients with autoimmune disease and inflammatory disorders.

The symptoms of autoimmune disease can be any abnormal symptoms resulting from or associated with inappropriate immune response of the body against substances and tissues normally present in the body. For example, the immune system mistakes “self” as foreign or a pathogen and destroys its own cells. The immune symptoms can also be any abnormal symptoms that are attributed to the generation of auto-reactive B and/or T cells. For example, auto-antibodies are a common symptom associated with autoimmune disease.

Of course, the symptoms of autoimmune disease may vary depending on internal or external factors presented to a patient. For example, the symptoms may depend on which ailment the patient suffers from. However, one skilled in the art may understand that there is a set of “super symptoms” that present in nearly every autoimmune disease. These symptoms are recognized indicators of autoimmune disease. The general symptoms include, but are not limited to, joint inflammation, muscle weakness, joint and muscle pain, skin lesions/rashes, fatigue, swollen glands, fever, dry eyes/dry mouth, malaise, and susceptibility to infection.

The course of the SU5416 treatment can be determined by the stage of the autoimmune disease or whether the infection is acute or chronic. Preferably, SU5416 is used in an acute therapy when clinical symptoms of autoimmunity or autoimmune diseases are diagnosed.

The effective amount of SU5416 for treating, reducing or alleviating symptoms of autoimmune disease and inflammatory disorders is not limited by the degree of benefit achieved by the administration of SU5416 in response to autoimmunity. For example, an amount of SU5416 may be considered effective where all symptoms are reduced, alleviated or even eliminated after the treatment. An amount of SU5416 may be effective if it reduces some symptoms but not all symptoms of autoimmune diseases appeared to a patient. An amount of SU5416 may also be effective if it delays the onset of at least one symptom.

In one preferred embodiment, SU5416 treatment of autoimmune disease would reduce or alleviate at least one of the symptoms of autoimmune disease including, but not limited to, those listed above. The reduction could be at least 10%, 20%, 30%, or preferably 40% if an effective amount of SU5416 is administered. For example, a patient diagnosed with psoriatic arthritis having undergone treatment with SU5416 might typically expect reduction in size and severity of psoriatic skin lesions by at least 10%. This patient might also experience mitigation of inflammation resulting in at least 10% reduction in joint stiffness and pain as a result of treatment with SU5416.

The duration of the treatment with SU5416 may vary depending on the relief progress of the symptoms of autoimmune disease in a patient. Typically, the treatment may last for a few weeks to months. Specifically, the treatment can be one week, two weeks, three weeks, four weeks, five weeks, six weeks, seven weeks, eight weeks, three months, four months, five months, six months, seven months, eight months, nine months, ten months, eleven months or twelve months. Treatment of SU5416 can be discontinued once a desired benefit is achieved.

The acute therapy of SU5416 can be followed by maintenance therapy, which is under different medications or treatments and may be given over the entire life of a patient. The acute therapy of SU5416 can also be followed by an induction therapy of different medications or treatments which is given to prevent the infection from occurring.

SU5416 would be administered either on its own or as part of treatment protocol. For example, SU5416 can be combined with other autoimmune treatments. SU5416 therapeutic composition can also be used in a combination therapy or serve as a supplemental component of an immunosuppressant cocktail that is currently used to treat these diseases.

The combination therapy with SU5416 can provide improved clinical benefits to a patient of an autoimmune disease by reducing the levels of antibody-producing and antigen-presenting B cells while rebalancing the T cell response toward a regulated state of T cell activation. The combination therapy may also help drive T-cell differentiation toward a more robust T_{reg} population. As one skilled in the art will understand, the exact mode and method of administration of the combination therapy may vary depending on the characteristics of the particular antibodies for T cells and B cells, and it also varies on particular patients and particular diseases.

In one preferred embodiment, SU5416 is administered with drugs that can enhance immunodulation. Thus, the combination may provide a synergistic therapeutic effect by interrupting, modulating or otherwise adjusting the signaling events that lead to T cell activation and the subsequent proliferation and differentiation of the T cell into effective T cells. Likewise, the combination therapy may provide a synergistic therapeutic benefit by interrupting, modulating or otherwise adjusting the signaling events that result in differentiation of an antigen presenting B cell into an antibody producing cell.

For example, SU5416 can be used together with T cell depletion agents or TNF inhibitors. Non-limiting examples of the drugs that can be used in combination therapy with SU5416 include Thymoglobulin (rATG), Campath, costimulatory blockade, infliximab, etanercept, adalimumab, golimumab, natalizumab, cytokines IL-10, IL-2, TGF-β, NSAIDs, corticosteroids, immune suppressants and TNF-α inhibitors. For example, if used together, both SU5416 and costimulatory blockade work synergistically to block secondary signaling of T cells an also enhance regulatory T cells.

The dose and method of SU5416 treatment will be similar to currently known SU5416 treatments for cancer. However, one will understand that the dose and treatment protocol may need to be specifically optimized for autoimmune disorders. For example, the dose may be the lower daily dose described above.

In some embodiments, SU5416 dosing follows the dosing used in Phase III clinical trials for the treatment of solid cancers. This dosing was found to be well-tolerated by patients and have acceptable side affects. For example, the administration of SU5416 might typically take place once or twice a week through IV infusion or central line for at least 6 weeks. The dose of twice weekly administration of SU5416 would preferably range from 85-145 mg/m²′. In a preferred embodiment, a patient receiving SU5416 though central line would receive 1 mg of COUMADIN daily as a prophylaxis against thromboemboletic events [64]. The first infusion of SU5416 or a therapeutic composition of SU5416 would typically be administered slowly (100 cm³/h) for the first 15 minutes before the infusion was increased to full speed (200 cm³/h) in order to reduce the likelihood of hypersensitivity reactions.

Patients would typically receive the SU5416 diluted in a solution such as KOLLIPHOR® EL and would typically be premedicated with an antihistaminic and dexamethasone to prevent the aforementioned hypersensitivity response.

These dosing strategies would be used as a starting point but may be modified to better suit the patient and the specific autoimmune disease being treated. These doses are likely to be higher than necessary. This is the highest dose that is well-tolerated in end-stage cancer patients and is probably the outer limit of dosing for this drug. In the case of autoimmune disease and inflammatory disorder treatments, we postulate a lower dosage, such as 5 mg/m², may be effective. A lower dosage would also reduce the potential of hypersensitivity responses and side effects.

### Transplant Rejection

In another embodiment of the present invention, SU5416 is a drug used to prevent or treat transplant rejection through treatment with post operative or preoperative dosing of an effective amount of SU5416.

Organ and tissue transplantation is the moving of an organ or tissue from one body to another or from a donor site on the patient's own body, for the purpose of replacing the recipient's damaged or absent organ. Transplant rejection occurs when transplanted tissue is “rejected” or deemed not “self’ and attacked by the recipient's immune system, which destroys the transplanted tissue.

Generally, transplant rejection is prevented or alleviated by use of immunosuppressant drugs after transplant. Immunosuppressive therapies vary; one such therapy uses short, repeated high dose courses of corticosteroids. Another therapy, “triple therapy”, adds a calcineurin inhibitor and an anti-proliferative agent to the corticosteroid regimen. Additional additives to these treatments can include antibodies to select immune components to further suppress the immune response.

The SU5416 treatment disclosed herein can be used to treat transplant rejection in a number of solid organ transplants including, but not limited to, lung transplants, skin transplants, cases of bronchiolitis-obliterans syndrome (BOS), heart transplants, liver transplants, kidney transplants, pancreas transplants, and corneal transplants.

In some embodiments, SU5416 is used as a precautionary treatment against transplant rejection for solid organ and skin transplant situations.

In some embodiments, SU5416 is used to treat acute or chronic transplant rejection, or both. Preferably, SU5416 is used in an acute therapy when the symptoms of rejection have begun.

The symptoms of transplant rejection may depend on the transplanted organ or tissue. Typically, these symptoms are characterized by loss of organ function. For example, a kidney rejection would be indicated by a rising creatinine level in blood and by means of a biopsy. Heart rejection is indicated by an endomyocardial biopsy, while pancreas rejection is determined by biopsy and rising blood glucose. Liver rejection is indicated by measurements of transaminases of liver origin, bilirubin levels in blood and by biopsy. Intestine rejection is determined by biopsy, while lung rejection is determined by measurement of blood oxygenation.

In the case of acute rejection, the tissue or organ becomes inflamed as the immune system attacks the organ. The goal of SU5416 treatment for acute rejection is to silence or quiet the immune response to the transplanted organ before substance damage to the organ occurs. Acute rejection occurs to some degree in many transplant patients. We foresee treatment with SU5416 preoperatively and post-transplant to reduce or prevent acute rejection and the damage the new organ incurs from the patient's immune system.

The SU5416 treatment for acute rejection may take place over anytime after the transplantation is done and the symptoms of rejections are realized. Typically, the symptoms of the acute rejection may starts from the first week after the transplantation to the third month.

The SU5416 treatment may last from a few weeks to months. Specifically, the treatment may last one week, two weeks, three weeks, four weeks, five weeks, six weeks, seven weeks, eight weeks, three months, four months, five months, six months, seven months, eight months, nine months, ten months, eleven months or twelve months. The SU5416 treatment may be stopped once the symptoms are adequately treated.

An “effective amount” of SU5416 for treating transplant rejection can be determined in various ways. For example, an amount of SU5416 is effective if it can inhibit or suppresses at least one symptom associated with transplant rejection. An amount is effective if it can delay the onset of and/or reduce the severity of at least one symptom associated with transplant rejection. Specifically, an effective amount of SU5416 can reduce or inhibit at least one symptom by at least 10%, 20%, 30%, or preferably 40% when compared to symptom before the treatment.

SU5416 is administered either on its own or with other therapeutic medications in a combination therapy. For example, other medications that can be used with SU5416 to treat transplant rejection are the medication that also drive T-cell differentiation to a more robust T_{reg} population (for example, IL-2 or IL-10). SU5416 or its composition can also be used as a supplemental component of an immunosuppressive cocktail that is currently used to treat these diseases. Non-limiting examples of the drugs that can be used in combination therapy with SU5416 include Thymoglobulin (rATG), Campath, costimulatory blockade, infliximab, etanercept, adalimumab, golimumab, natalizumab, cytokines IL-10, IL-2, TGF-β, NSAIDs, corticosteroids, immune suppressants and TNF-α inhibitors.

The dosing strategy of SU5416 in treating transplant rejection on its own or as a component of a combination therapy or an immunosuppressive drug cocktail would follow the dosing described above. These dosing strategies would be utilized as a starting point but would be modified to better suit the patient and the reactivity of their immune system. These prior doses are likely to be higher than necessary. This is the highest dose that is well-tolerated in end-stage cancer patients and is the outer limit of dosing for this drug. In the case of immunosuppressive treatments to prevent transplant rejection, we envision that a lower dosage may be effective; a lower dosage would also reduce the potential of hypersensitivity responses and side effects.

In some embodiments, the dosing strategy of SU5416 in acute rejection therapy would be similar or same to the dosing strategy in phase III clinical trials for late-stage cancer treatment. In response to acute rejection, treatment of the patient with a moderate to high dose (50-150 mg/m²) of SU5416, depending on severity of the rejection. The SU5416 could be administered to the patient as a stand-alone therapy or more likely as a component of an established treatment protocol. Also, SU5416 may also be used as a preoperative preventative treatment, where the patient would likely receive a low dose (potentially around 10-50 mg/m²) to establish a more tolerant immune environment.

In the case of chronic rejection, a consistent low dose treatment strategy of SU5416 post-transplant would typically be used to decrease immune activation for the transplanted organ. Specifically, a lower dose strategy equivalent to the lower ranges tested in Phase III cancer drug trails may be utilized. The higher dosing strategy would typically be used in combination with current immunosuppressive therapy to treat rejection and save the transplanted organ from destruction.

For example, in the case of a patient with bronchiolitis-obliterans syndrome (BOS), treatment with SU5416 on its own or as a component of an established treatment would alleviate shortness of breath. The alleviation of symptoms would be measured by an increase in forced expiratory volume in one second (FEV1) by five percent. FEV1 should be above 80% of predicted values to be considered normal. In cases of BOS, an individual's FEV1 is typically reduced to 16% to 21% of predicted values. We envision treatment of patients with BOS would typically comprise a moderate to high dose of SU5416 (50-150 mg/m²) and result in alleviation of wheezing and shortness of breath as determined by the patient and/or a 5% increase in FEV1.

### SU5416-Like Drug Discovery

In another embodiment of the present invention, SU5416 would be used as a template for drug discovery for additional methods of treating autoimmune diseases and transplant rejection. The method would comprise the steps of (a) examining target chemicals for structural and functional similarity to SU5416 and (b) identifying a chemical with sufficient functional similarity to SU5416 so that treatment with the chemical produces therapeutic results. In some embodiments, the step of (c) treating the autoimmune or transplant patient with an effective amount of the SU5416-like chemicals would be added.

We recognize the unique duality of SU5416 signaling and the benefits of this divergent signaling in immune modulation and its enormous potential as a drug of immune dysregulation. Therefore, SU5416 is a good chemical as a model for discovery of other drugs of this ilk. This could be accomplished through structure activity relationships (SAR). SAR uses the relationship between the chemical or 3D structure of a molecule and its biological activity. The analysis of SAR would enable the determination of the chemical groups responsible for evoking a target biological effect in an organism. This allows modification of the effect to the potency of a bioactive compound by changing its chemical structure.

### Compositions of the Present Invention

In the clinical trials, the patients were dosed with SU5416 once or twice a week through IV infusion or central line for at least 6 weeks. The dose of twice weekly administration of SU5416 ranged from 85-145 mg/m². In one embodiment of the invention, one would use these doses. We envision that, in another embodiment of the invention, an effective dose is lower. For example, one might use a daily dose of at least to 5 mg/m² up to the dose described above.

Patients in the clinical trials received SU5416 though central line would typically receive 1 mg of COUMADIN daily as a prophylaxis against thromboembolic events [64]. In one embodiment of the present invention, the patient would be treated with COUMADIN in a similar manner.

The first infusion of SU5416 or a therapeutic composition of SU5416 would typically be administered slowly (100 cm³/h) for the first 15 minutes before the infusion is increased to full speed (200 cm³/h) in order to reduce the likelihood of hypersensitivity reactions.

Preferably, patients would receive the SU5416 diluted in a diluent able to stabilize emulsions of nonpolar materials in aqueous systems, such as KOLLIPHOR EL.

Preferably, as in the clinical trials, all patients would be premedicated with an antihistaminic and dexamethasone to prevent against hypersensitivity reactions to the diluent.

In one embodiment, the present invention is a therapeutic composition containing an effective dose SU5416. In some embodiments the composition also comprises additional therapeutic agents.

For example, the additional agent may be costimulatory blockade. SU5416 and costimulatory blockade can work synergistically to block secondary signaling of T cells an also enhance regulatory T cells.

The additional agent may be cytokines. Cytokines can direct T-cell differentiation into a more robust T_{reg} or into a T_{effector} population to modulate the immune system. Different cytokines result in different populations which have there own advantages in immune-modulation and treatment of disease. For example, in most autoimmune diseases T_{reg} cells are advantageous as they create a more immune-tolerant environment and reduce the attack of “self” by native T-cells. We foresee treatment of autoimmune patients with SU5416 and other T_{reg} inducing cytokines, such as IL-2 and IL-10, resulting in an increase in the amount of T_{reg} cells and mitigation of disease symptoms.

SU5416 can also be combined with other known treatments for autoimmunity or transplant rejection to create effective drug compositions. For example, in treatment of patients with autoimmune disorders a suitable composition would be comprised of an effective amount of SU5416 and at least one of the following types of drugs: non-steroidal anti-inflammatory drugs (NSAIDs), corticosteroids, immune-suppressants, and TNF-α inhibitors. In the case of transplant patients a suitable composition would comprise an effective dose of SU5416 and at least one of the following types of drugs: calcineurin inhibitors, immunosuppressants, interleukin inhibitors, mTOR inhibitors, anti-proliferatives, and corticosteroids.

### Examples

The work disclosed in the Examples began with high throughput screening for AHR ligands at the University of Wisconsin-Madison: Small Molecule Screening Facility (UW SMSF). One of the hits was SU5416. It has been found that SU5416 is a strong ligand of AHR and the drug bound equally well to both the strong and weak forms of AHR, which is very unusual. It has also been found that SU5416 activates the IDO pathway in dendritic cells and learned that SU5416 enhances T cell differentiation to Regulatory T cells. The field understands that an increase in regulatory T cells has immunomodulatory effects that would be useful in transplants and in autoimmune disorders. Our recent data and understanding of these novel mechanisms make this drug an excellent candidate to be used to treat transplant rejection and/or autoimmune diseases.

Primary Screen for Agonists of the Human AHR.

To identify novel agonists of the AHR, a library of 4,160 small molecules, “The KBA library”, was screened at 10 μM per compound, by the Small Molecular Screening Facility of The Carbone Cancer Center of the University of Wisconsin School of Medicine and Public Health. This library represents the sum of three commercially available well characterized chemical libraries with a high frequency of approved drugs and prototype signaling molecules. This includes 2,000 diverse FDA approved drugs and natural products (Microsource Discovery Systems, Inc; Gaylordsville, Conn.); the 1280 compound LOPAC¹²⁸⁰ library of diverse characterized compounds (Sigma; St Louis, Mo.); and 880 characterized compounds (Prestwick Chemicals; Illkirch, FR).

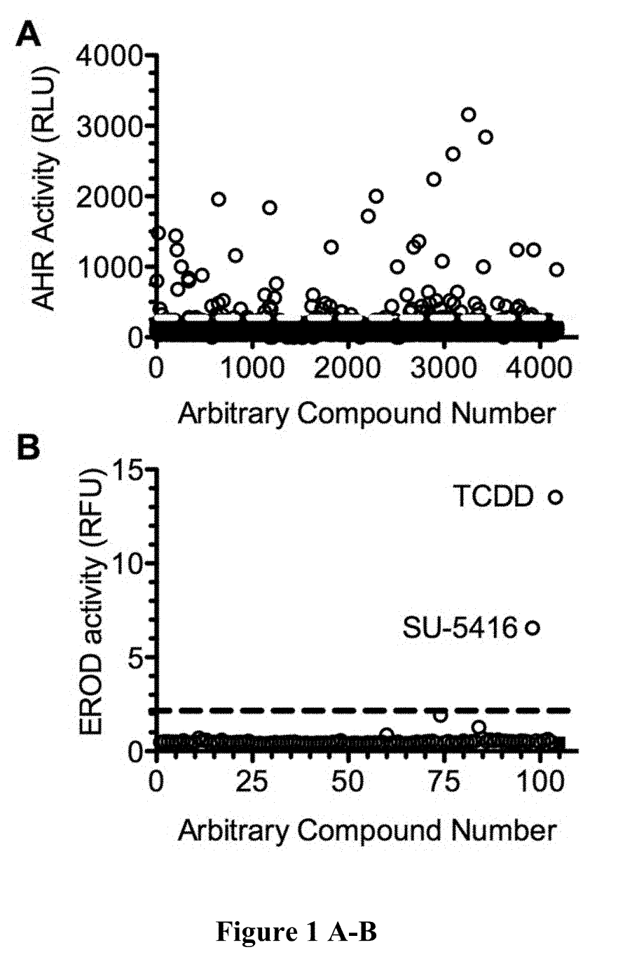

In this first stage of the screen, AHR agonism was determined by monitoring the activation of the human receptor using the human 101L-hepatoma cell line that has a stably integrated “dioxin-responsive element (DRE) driven luciferase reporter [13]. At the tested concentration of 10 μM, approximately 100 compounds induced at least a three-fold increase in luciferase activity (FIG. 1A).

Secondary Screen for Agonists of the Murine Ahrd Low Affinity Receptor.

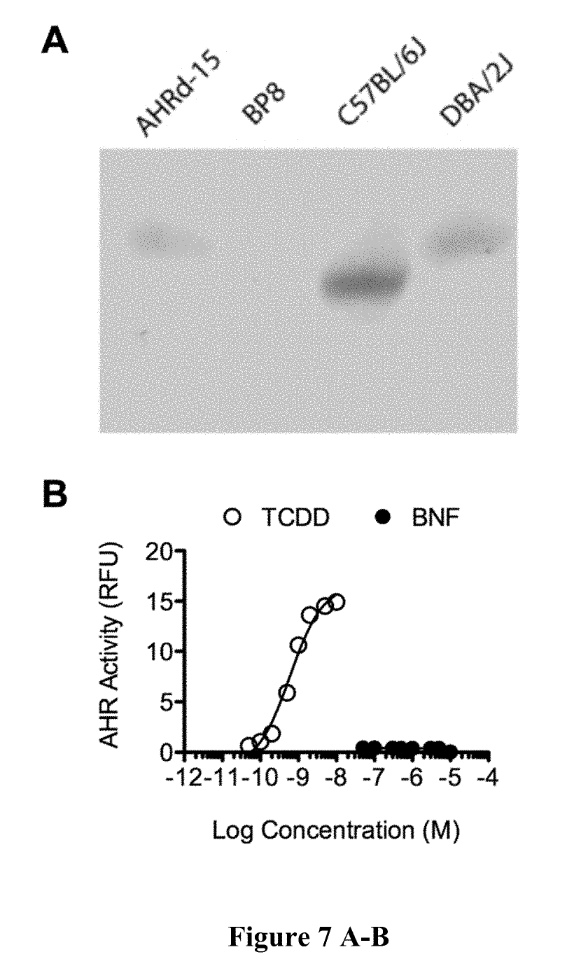

The 100 “hit compounds” from the primary screen were subsequently screened for their capacity to activate the low-affinity murine AHR^{d} receptor isoform using the activity of the endogenous Cyp1a1 gene as a readout. To this end, we established a hepatoma cell line that expresses the AHR^{d} receptor isoform derived from the DBA/2J mouse[14]. An AHR^{d}-expressing cell line was generated by stably transfecting the AHR^{d} cDNA into the rat hepatoma AHR-deficient cell line, BP8[15]. After stable selection with G418, a sub clone (AHR^{d}-15) was analyzed for receptor expression and function. First, a western blot using an anti-AHR antibody, revealed that the AHR^{d}-15 cells produced an immune-reactive protein band that co-migrated with a receptor species isolated from the hepatic cytosol of DBA/2J mice (approximate size 104 kDa). This band was distinct from the AHR^{b} isoform found in C57BL/6J cytosol, which migrated at 97 kDa (FIG. 7A). To confirm that the AHR^{d}-15 clone expressed a functional low affinity AHR^{d} isoform, we examined the receptor-mediated response to the prototype agonists, TCDD and β-naphthoflavone (BNF). Increasing concentrations of TCDD induced CYP1A1-mediated EROD activity in these cells with an EC₅₀ in the 30 nM range [16]. In contrast, the much weaker agonist, BNF, known not to induce an AHR-mediated response in the AHR^{d} receptor isoform expressed in the hepatocytes of DBA/2J mice[17] was shown to be inactive at doses as high as 10 uM in the AHR^{d}-15 cells (FIG. 7B).

To test the ability of the 100 AHR inducers to activate the AHR^{d}-15 cells, they were treated with each of the compounds at the dose of 1 μM, for 36 hours in 96-well plates. Only the compound SU5416, and the positive control, TCDD, induced AHR^{d}-mediated EROD activity greater than 5-fold (FIG. 1B). Therefore, SU5416 was considered for further analysis.

Induction of DRE-Mediated Transcription by SU5416 is AHR and ARNT Dependent.

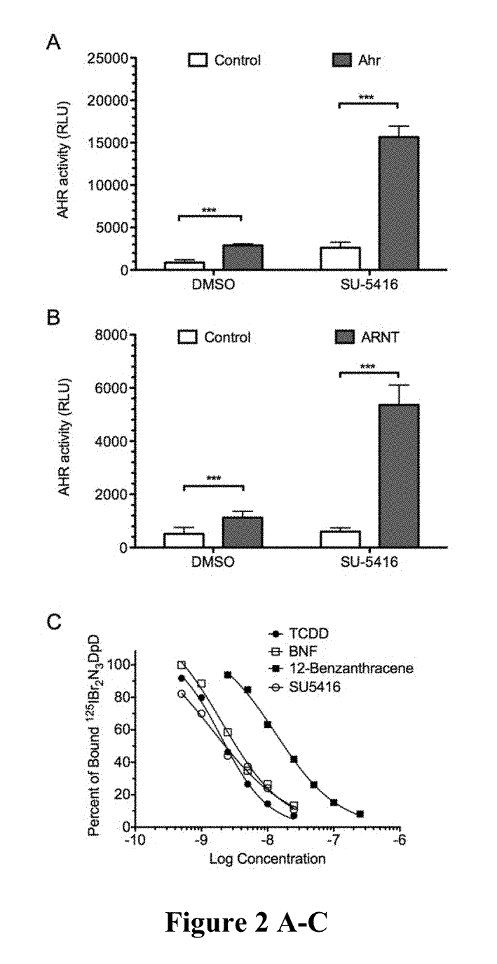

To prove that induction of the DRE was mediated through classic AHR signal transduction, and not through a VEGF-related mechanism, we employed mutant cell lines that lack expression of the AHR or ARNT. The C35 cell line, which contains a dysfunctional AHR, was utilized[18]. It was transfected with vector containing the murine AHR gene, the lacZ gene, and the luciferase reporter gene driven by 3 upstream DREs, as described in the Methods section. Controls were mock transfected with reporter plasmids and the empty vector. Cells were treated with either 3 μM SU5416 or DMSO (control). As seen in FIG. 2A, cells transfected with the AHR plasmid generated significant luciferase activity when exposed to SU5416 compared to DMSO. The control cells generated minimal activity.

In a similar experiment, the ARNT-deficient mouse hepatoma cell line C4 was transiently transfected with plasmids encoding human ARNT, the lacZ gene, and the same DRE-driven luciferase gene, and control samples received empty vectors for ARNT[19,20]. As shown in FIG. 2B, after exposure to SU5416 or DMSO, activity was only seen when ARNT was transfected.

SU5416 is a Ligand of the AHR.

To confirm that this molecule is a direct ligand of the AHR and not working through some other agonist, we performed competitive binding assays of the AHR using a radioligand. Photoaffinity experiments incubating ¹²⁵Ibr2N3DpD with the hepatic cytosolic fraction from C57BL/6J mice (AHR^{b} isoform) were conducted as described in the Methods[21]. Increasing concentrations of SU5416, TCDD, BNF, and 1,2-Benzanthracene (a ligand of low receptor affinity) were added. As shown in FIG. 2C, SU5416 competitively displaced the radiolabel with efficacy similar to TCDD.

In Utero Exposure to SU5416 Stimulates Closure of DV.

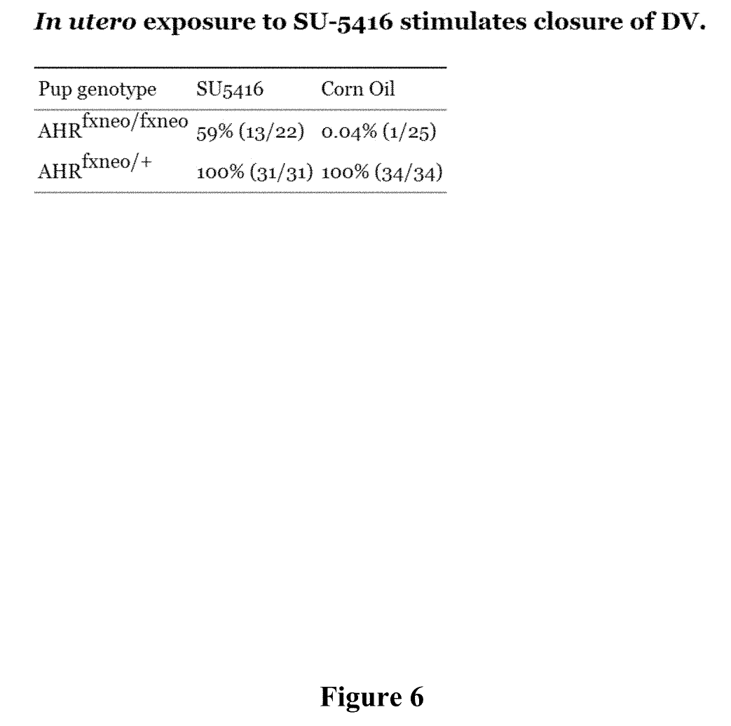

We have previously shown that genetically altered mice that express only 10% of the AHR display a patent ductus venosus (DV) in the liver in nearly all cases [22]. We additionally identified that in utero activation of the receptor in the hypomorphs with TCDD successfully closed the DV[5]. To test the role of SU5416 as an in vivo ligand and its potential effect on embryology and vascular development, we performed timed matings of female AHR^{fxneo/+} mice to male AHR^{fxneo/fxneo} mice. The pregnant dams were treated at embryonic day E18.5 with a single dose of SU5416 at 110 mg/kg, or an equivalent volume of the vehicle, corn oil. At 4 weeks of age, the pups were sacrificed, and DV status was examined by hepatic perfusion with trypan blue. As seen in FIG. 6, only 1 of 25 AHR^{fxneo/fxneo} pups treated with corn oil possessed a closed DV. In the experimental group, 13 of 22 animals of this phenotype exposed to SU5416 had a closed DV.

SU5416 Up Regulates CYP1A1 and CYP1B1.

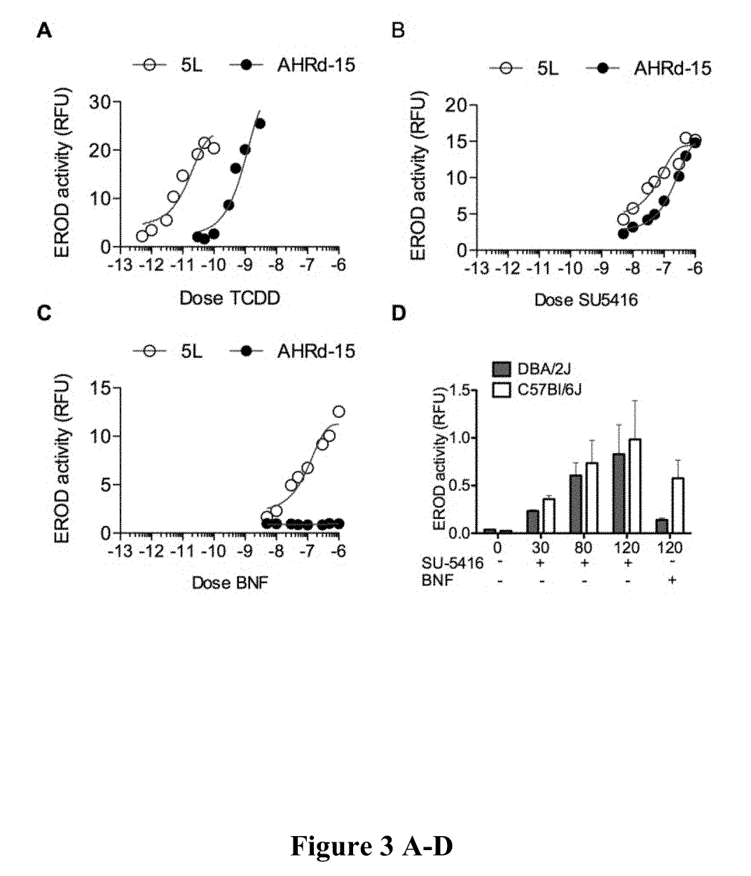

The above data clearly shows that SU5416 is a ligand of the AHR. We now focused our attention on the strong response of SU5416 to the AHR^{d} polymorphism in the screening assay, and compared the activity of this ligand in the high and low affinity polymorphisms. We utilized the wild type rat hepatoma cell line, 5L, which harbors the high affinity AHR isoform, and our newly created AHR^{d}-15 cell line. As seen in FIG. 3A, we first performed a titration with TCDD and measured EROD activity. As expected, the activity of TCDD was shifted by 1.5 orders of magnitude to the left for the AHR^{b} isoform. In contrast, when SU5416 was tested in vitro, the two curves virtually overlapped (FIG. 3B), showing equal potency for cytochrome P450 induction using the two cell lines. We also tested BNF, which as expected, showed a strong response with the 5L cell line and no response with the AHR^{d}-15 cell line (FIG. 3C).

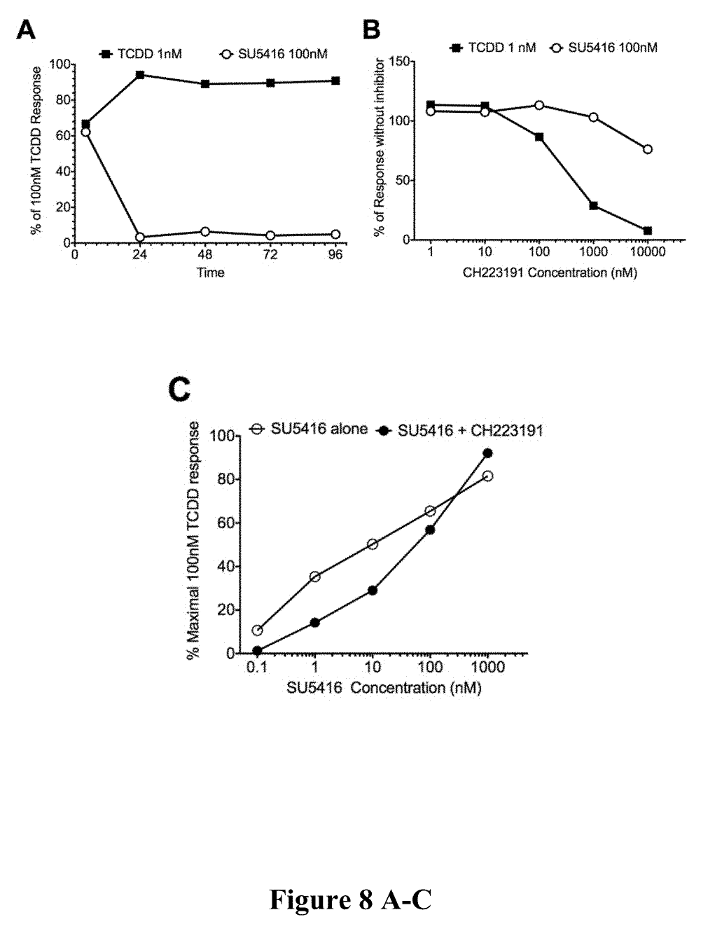

As these experiments were done in cell lines, and in addition the AHR^{d}-15 line combines a rat cell line with a transfected murine AHR, we further tested the ability of SU5416 to activate the AHR in vivo. Six-week old C57BL/6J mice (AHR^{b}) and DBA/2J (AHR^{d}) were orally administered 30, 80, or 120 mg of SU5416 per kg of body weight. Groups of control mice were given corn oil alone or BNF at the concentration of 120 mg/kg. Treatment of SU5416 produced a dose-dependent increase in EROD activity in both strains of mice seen after sacrifice (FIG. 3D), with no significant difference between the two. BNF at 120 mg/kg showed dramatically decreased in vivo activity in the DBA/2J strain. To identify the duration of activity of SU5416 as a ligand of the AHR, we dosed human 101L-hepatoma cells with SU5416 at a dose of 100 nM or TCDD at 1 nM, and measured luciferase activity at 4, 24, 48, 72, and 96 hours. As can be seen in FIG. 8A, SU5416 has activity at 4 hours, but by 24 hours no longer causes luciferase activity indicating loss of binding to the DRE, which is clearly in contrast to the long duration DRE-binding seen with TCDD. Of note, when we did titrate SU5416 doses as high as 10 μM, we did observe as much as 20% of TCDD response (1 nM) as far out as 96 hours (data not shown). This SU5416 data is similar to the known plasma half-life of 30 minutes, although VEGF-receptor inhibitor effects have been shown to last as much as 72 hours in culture[23].

We further analyzed whether the AHR antagonist CH223191 could inhibit the ability of SU5416 to activate the DRE in 101L-hepatoma cells. It has previously been shown that this antagonist inhibits TCDD but not some of the other ligands of the AHR including some polycyclic aromatic hydrocarbons. We first performed a titration of the AHR antagonist in culture with either 1 nM TCDD or 100 nM SU5416. As can be seen in FIG. 8B, the effects of TCDD are inhibited whereas minimal inhibition is shown for SU5416. In FIG. 8C, we show a titration of SU5416 with only a small amount of inhibition of activity by the inhibitor.

SU5416-Induced Up Regulation of CYP1A1 is Similar in Murine AHR^{b} and AHR^{d} splenocytes.

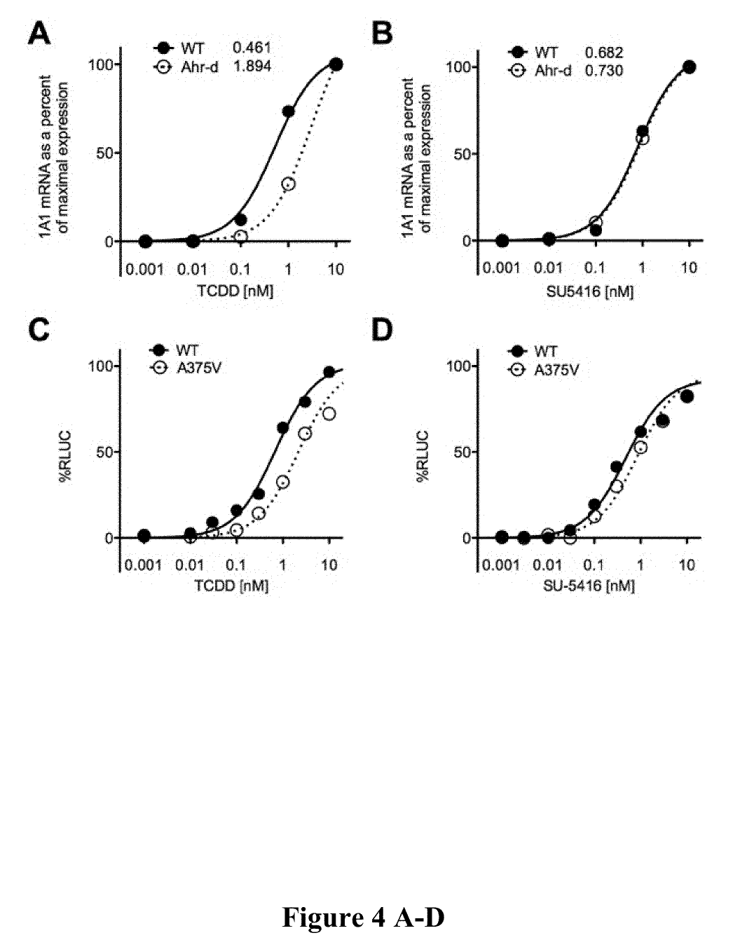

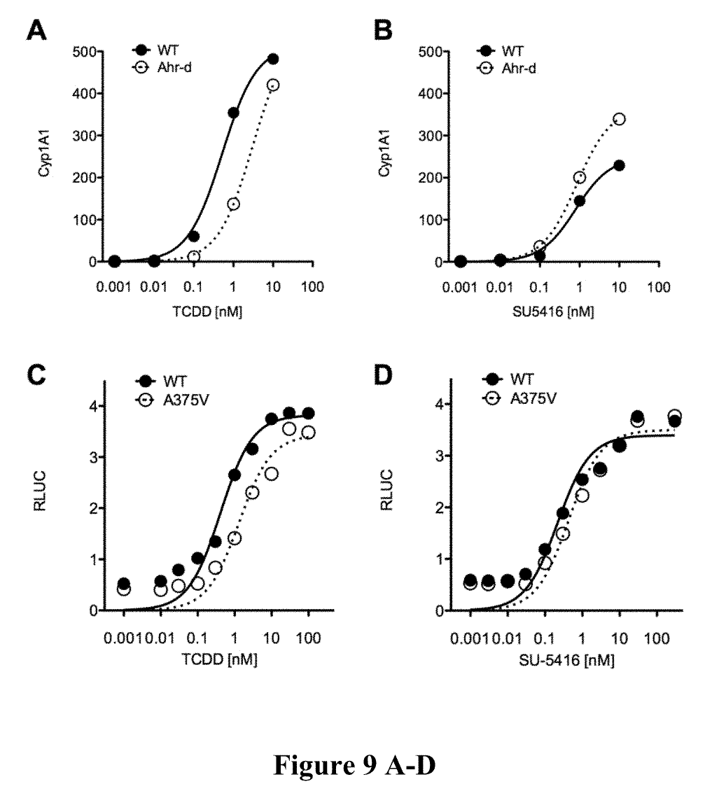

As the above in vitro experiments were performed in cell lines, we next utilized AHR^{b} (C57BL/6J) and AHR^{d} congenic mice (on a C57BL/6J background). Spleens from these mice were harvested and suspended in culture media, and exposed to titrating doses of TCDD and SU5416. These data are presented in FIG. 4, where the graphs show normalized data from 0 to 100% response. Normalized data was chosen to allow comparison of CYP1A1 up regulation to its maximum in AHR^{b} versus AHR^{d} mice. After 4 hours of culture, TCDD induced CYP1A1 more rapidly and to a higher degree in wild-type than AHR^{d} splenocytes, with an EC₅₀ of 0.461 nM in wild-type and 1.894 nM in AHR^{d} animals. FIG. 4B shows that SU5416 induced CYP1a1 similarly in AHR^{b} and AHR^{d} mice, with an EC₅₀ of 0.682 nM in wild-type and 0.730 nM in AHR^{d} mice. FIGS. 9A and B show the total fold change seen by qPCR analysis of splenocytes after exposure to TCDD and SU5416, to allow an assessment of the potency of AHR activation of these two ligands with CYP1A1 induction as the readout. As can be seen in the Fig., TCDD elicits more CYP1A1 in AHR^{b} compared to AHR^{d} mice, whereas SU5416 leads to the same or more CYP1A1 in AHR^{d} mice. By this readout, TCDD and SU5416 have similar potency in AHR^{d} cells, and TCDD is a stronger ligand in AHR^{b} cells.

Cos-1 Cells Transfected with AHR Containing Valine Point-Mutation Show Similar ED₅₀ to Cos-1 Cells with AHR^{b} Isoform.

Because of the importance of identifying that SU5416 is truly unique in its ability to activate the low affinity AHR isoform with similar strength as the high affinity isoform, we transfected COS-1 cells with an AHR containing the same point mutation (valine for alanine) thought to be responsible for the low affinity of the AHR^{d} isoform compared to AHR^{b}. These cells also harbor a luciferase gene next to the DRE. We again tested SU5416 and TCDD, harvesting the cells after 4 hours. As seen in FIG. 4C, the ED₅₀ for TCDD was higher in the A375V transfected cell line (AHR^{d} type) compared to wild-type, with a much smaller difference between the two isoforms for SU5416 (FIG. 4D). Specifically, the EC₅₀ for TCDD was 0.73 nM in wild-type cells, and 2.47 nM in transfected cells, while for SU5416 the EC₅₀ was 0.18 nM in wild-type, and 0.31 nM in transfected cells. The EC₅₀ is actually lower for SU5416 than TCDD in either cell line, supporting that this is a potent and unique ligand of the AHR that has only mild loss of binding capacity when faced with the valine point mutation see in the AHR^{d} isoform. The data in this Fig. is normalized from 0 to 100% response. The actual luciferase values are included in FIGS. 9C and D, which again show the potency of these two ligands.

SU5416 Leads to IDO Induction and FoxP3 Up Regulation in CD4⁺ T Cells.

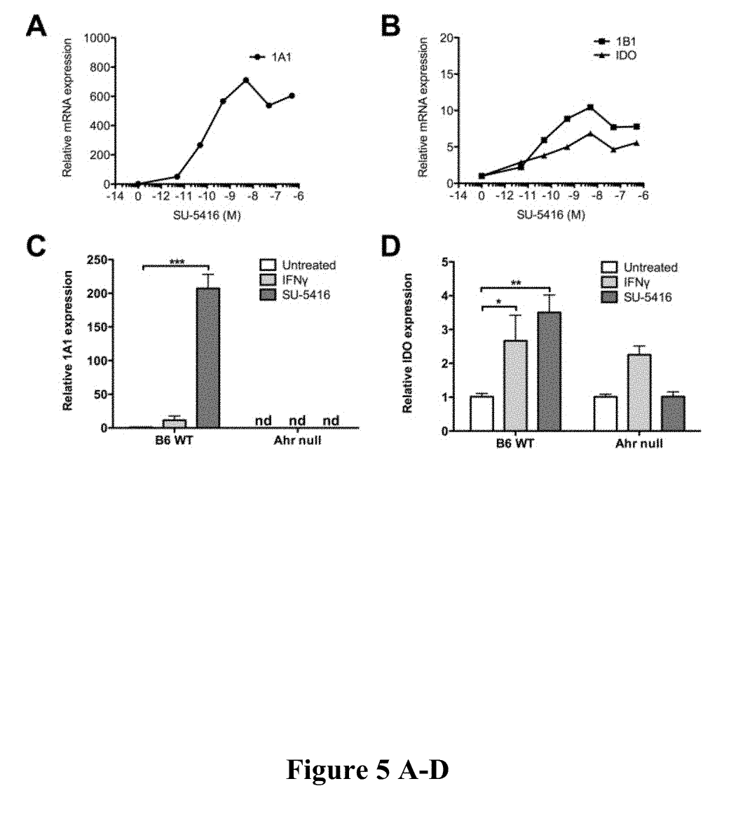

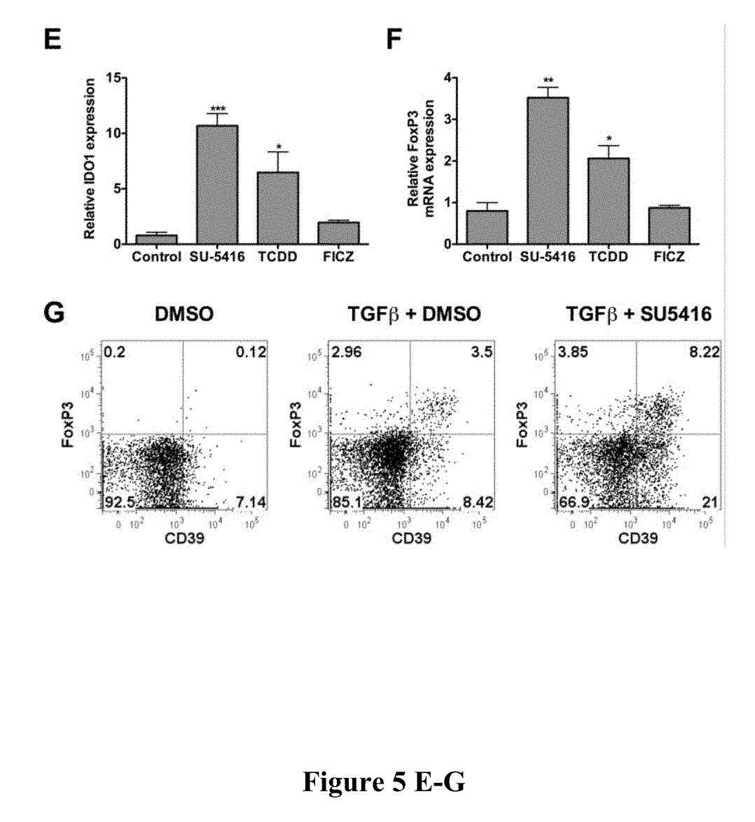

An important role for the AHR in the immune system, and specifically T-cell differentiation, has been recognized and continues to be characterized in the literature[6,7]. Some ligands of the AHR have the ability to enhance Treg differentiation from naïve T-cells (TCDD, kynurenine), while others direct differentiation towards Th17 effector cells (FICZ). We first tested the ability of SU5416 to induce CYP1A1 and CYP1B1 when titrated in solution with cultured splenocytes. Spleens from C57BL/6J mice were harvested and suspended in culture media, and exposed to titrating doses of SU5416. As seen in FIG. 5A, after 4 hours of culture SU5416 dramatically induced these cytochrome P450 enzymes in a dose-dependent manner, indicating activation of the DRE in vitro.

In this same assay we tested the ability of SU5416 to generate the CYP1B1 and the enzyme IDO, the first enzyme in the kynurenine pathway of tryptophan metabolism. IDO has long been known to play a role in Treg generation, and may be central to the mechanism of Dendritic Cell (DC)-directed Treg generation[24]. We as well as others have previously shown that IDO mRNA can be induced by ligands of the AHR, and that the mechanisms of DO-directed Treg generation may depend on the AHR[25]. This assay shows that SU5416 induced significant amounts of IDO mRNA in splenocytes (FIG. 5B), a finding that was previously reported for TCDD[26]. To confirm that CYP1A1 and IDO induction in splenocytes is in response to an interaction with the AHR and not secondary to an interaction with the VEGF receptor, we compared the response of AHR wild-type and AHR^{-/-} cells to SU5416 and IFN-γ. As shown in FIGS. 5C and D, SU5416 induced CYP1A1 in wild-type but not null cells. Additionally, IDO was induced by SU5416 in wild-type but not null cells, confirming the importance of this receptor in IDO induction. IFN-γ did lead to some IDO induction in both wild-type and null cells (although it did not reach statistical significance in the null cells in this representative assay), a finding we have seen in prior experiments [25].

To assess if FoxP3 could be generated by SU5416 exposure, we employed a pDC/T cell co-culture. Previous authors have suggested that Treg generation in this assay is driven by IDO production by the plasmacytoid DCs (pDCs)[27]. As described in the Methods, naïve T-cells were sorted using magnetic bead separation, and placed in culture for 5 days with allogeneic pDCs separated from BALB/C mice. SU5416, TCDD, FICZ, or media alone was added at the start of culture. After 5 days, cells were collected and mRNA harvested for qPCR analysis of IDO and FoxP3. As shown in FIGS. 5E and F, IDO and FoxP3 were generated after addition of SU5416 in this assay. This up regulation was also seen with TCDD, which has been previously reported to induce FoxP3 [6]. In order to look at the direct effect of SU5416 on T cells alone, we separated naïve CD4⁺ T cells and exposed them to TGF-β with or without SU516. As can be seen in FIG. 5G, the addition of SU5416 significantly enhanced the FoxP3 protein expression by flow cytometry.

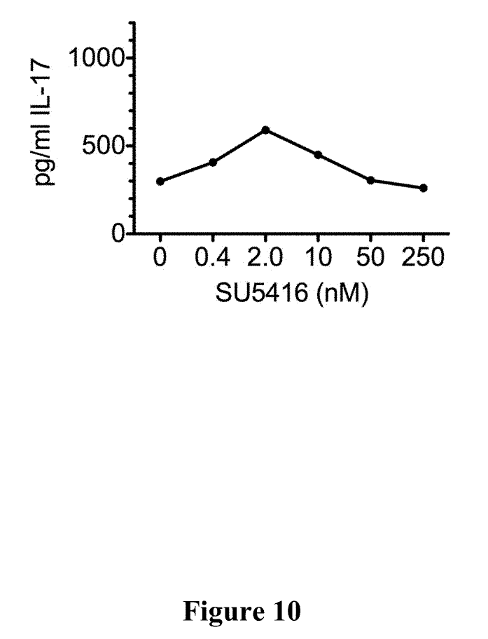

To further support that SU5416 leads to regulatory cells, we also analyzed the up regulation of CD39, which is an ectoenzyme that degrades ATP to AMP and is strongly associated with Tregs that can suppress ATP-related effects and pathogenic Th17 cells. As can be seen in FIG. 5G, SU5416 up regulated CD39 in the FoxP3⁺ T cells, a finding that has recently been reported with TCDD[28]. Finally, as literature is emerging that the ability of AHR ligands to enhance T-cell differentiation may be dependent as much on surrounding conditions and inflammatory milieu as on the ligand tested, we assessed the ability of SU5416 to enhance Th17 differentiation in Th17 conditions. Naïve T cells were placed in culture with IL-6 and TGF-β, and harvested after 3 days of culture. FIG. 10 shows that at low doses SU5416 caused a small increase in IL-17 protein by ELISA in the supernatant. At higher doses we did not see this effect.

Skin Grafts.

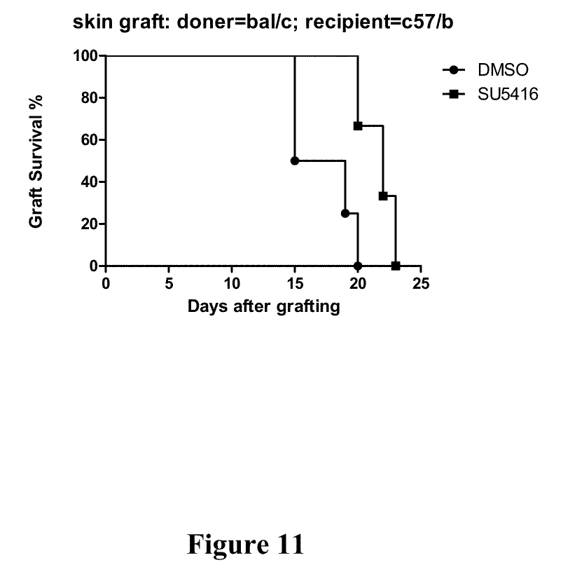

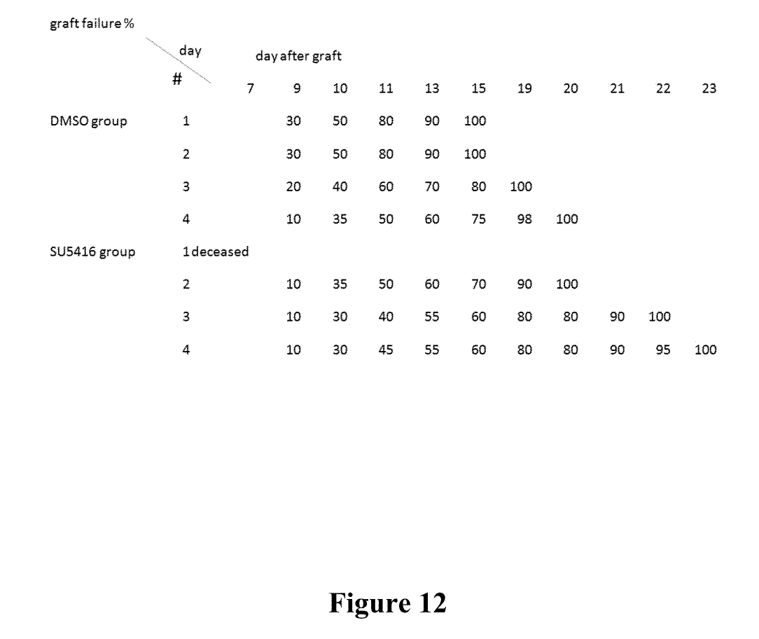

Four C57BL/6 mice received skin grafts from fully mismatched Balb/C donors. In this strain combination, the mice were fully mismatched at their major histocompatibility complexes and reject quickly without immunosuppression. Four of the mice were treated with DMSO, which is simply the solvent that SU5416 is dissolved in (and serves as the control). Four mice received 2 doses of SU5416 (50 mg/kg, given 2 and 5 days after skin grafting). One animal in the SU5416 group died unexpectedly from anesthesia. FIG. 11 shows a survival curve, where rejection of each skin graft is identified by a vertical drop in the survival line. FIG. 12 shows a chart of scoring of the skin grafts. It can be seen from these figures that mice that received SU5416 had prolonged graft survival compared to the control group.

Comparison of Abilities to Activate the AHR Between VEGFR02 Kinase Inhibitors.

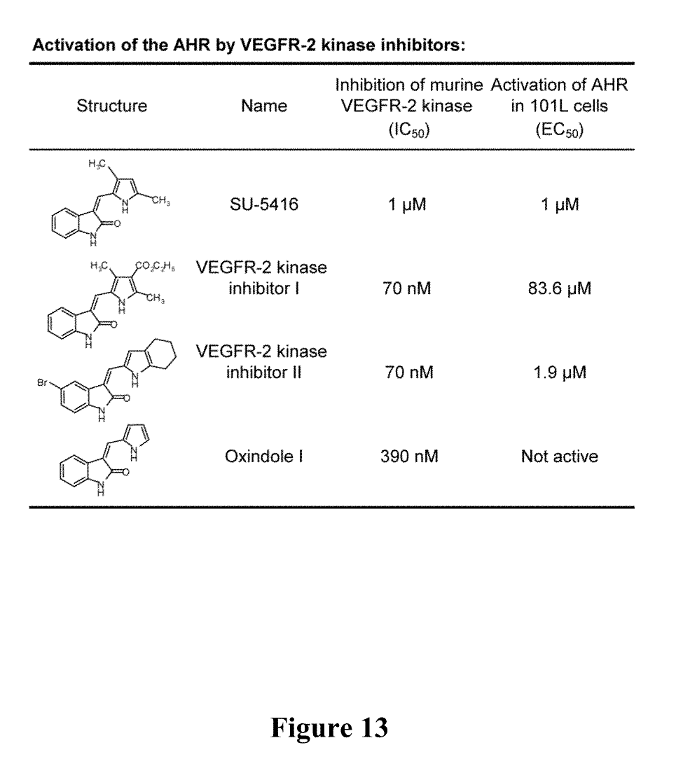

FIG. 13 shows an example of the test comparing three VEGFR-2 kinase inhibitors on their abilities to activate the AHR. As can be seen in the table, the ability to activate the AHR did not correlate to the ability to inhibit VEGFR-2. One of the strong VEGFR-2 inhibitors did not activate the AHR at all, and the other inhibitors all had variable strength of activation of the AHR.

### Discussion

It has been demonstrated recently that aryl hydrocarbon receptor (AHR) plays a role in immunology in addition to the expected role as a hazard response for the cell. The role of AHR in immunology has been emphasized by reports that activation of this receptor with ligands, such as TCDD, can lead to the generation of regulatory T-cells (Tregs) [6], while activation with other ligands, such as formylindolo[3,2-b]carbazole (FICZ) can lead to Th17 cell formation [7]. T-reg and T-effector cells are important modulators of the immune system with opposing roles, thus making AHR a dynamic player in the immune response. Manipulation of AHR could be a particularly powerful tool in regulating the immune system.

In the present invention, a library of compounds with known biological activity (KBA) was screened for AHR activity, to identify agonist to be used as drugs for AHR immune modulation. [3-(3, 5-dimethyl-1H-pyrrol-2-ylmethylene)-1,3-dihydro-indole-2-one] (SU5416) was identified as a strong agonist of both isoforms of AHR. SU5416 is a known inhibitor of VEGFR2 that was initially designed as a cancer drug; SU5416 progressed to phase II clinical trials for the treatment of metastatic colorectal cancers. It was well tolerated and showed promise but its lack of efficacy led to its abandonment. The inventors observation that SU5416 is, in addition to a strong inhibitor of VEGFR2, a strong agonist of AHR provide a new understanding of this drugs effects and suggest an answer to the lack of efficacy seen in the cancer trails. The dual signaling of SU5416 has implications for future clinical trials and provide a promising direction for future efforts aimed at diseases particularly well suited for such a pharmacologically unique compound.

Our recognition that the agonist of AHR can have an impact on the immunological out come of this signaling, make AHR an interesting target for treatment of diseases including autoimmunity and transplant rejection. Paradoxically, also potentially for cancer therapy, as it was initially designed, depending on the ligand employed. Based on efforts at characterizing novel ligands of the AHR in relation to their interaction with the acquired immune system, we envision that ligands can either be “regulatory” or “effector”, depending on the inflammatory milieu and dosing strategies of the ligands. This is the inventors conceptual basis for developing this drug and others like it as an entirely new class of drugs targeting the AHR for immunomodulation.

Examples above demonstrate the ability of SU5416 to activate the AHR^{b} and AHR^{d} polymorphisms with similar efficacy. These two isoforms are present in different strains of mice, and have been well characterized for many ligands, particularly TCDD. For the majority of ligands studied, the AHR^{d} isoform displays less than one-tenth the response of AHR^{b} after binding. SU5416 activates these two isoforms with similar potency. This not only confirms the importance of this property of the drug in humans, who harbor the AHR^{d} polymorphism, but also will allow the structure of SU5416 to serve as a model in our search for clinically relevant endogenous ligands of the AHR. This in turn can be used to discover or design new ligands/agonists that can be utilized as drugs or be part of a combination of compounds used as drugs for immune modulation, expanding the drug repertoire available for treatment of these diseases of immune dysregulation for which there are few truly effective treatments.

The present invention relates to treatment of disease through immune modulation by activation of aryl hydrocarbon receptor. The invention also relates to the AHR agonist SU5416 and manipulation of its dual properties applied to the treatment of immune diseases/disorders and transplant rejection. The invention also relates to the use of SU5416 to identify and/or design additional/more efficient agonists of AHR to be used in the treatment of disease.

SU5416 is a strong ligand of the AHR. The unique finding that SU5416 binds the high- and low-affinity polymorphisms of the AHR similarly was rather surprising to us, and will require further attention and characterization. The mouse AHR can arise from an allele that encodes a receptor with high binding affinity for ligand (denoted Ahrb allele) or with low binding affinity for ligand (denoted as the Ahrd allele). The AHR^{d} is known to have approximately one-fifteenth to one-twentieth the binding affinity to TCDD as the AHR^{b}[36], and this low affinity polymorphism resembles the isoform found in humans[37,38,39,40]. C57BL/6 mice harbor the high-affinity AHR^{b} receptor, and this strain has been utilized for much of the initial characterization of TCDD and other environmental toxicants[3]. In our search for relevant ligands of the AHR, we decided to focus on those that had significant potency in the AHR^{d} isoform, as these ligands would have more clinical relevance in humans. We inadvertently identified that SU5416 had similar binding characteristics with both polymorphisms at doses that are similar to what were used in humans in Phase I trials with SU5416[30], as seen in the titration in FIG. 3. This is an unusual characteristic that has rarely been exhibited by any of the known ligands of the AHR [41].

The importance of this is due to the following: First, the information is clinically significant given that humans harbor an AHR isoform that more similarly represents the AHR^{d}. Second, its structure will serve as a model in our search for endogenous ligands of the AHR. It makes sense that a true endogenous ligand would activate both polymorphisms of the AHR similarly, given that mice (and humans) that harbor the low affinity polymorphism do not exhibit the patent ductus venosus found in AHR nulls and hypomorphs. This is further supported by the ability of SU5416 to close the DV in AHR hypomorphs (a requirement of the true endogenous ligand)[12]. To this point we have been unable to model the binding sites of these polymorphisms by crystallography, but the finding that SU5416 can bind both of these similarly may help us in these efforts. At the very least, it confirms that a potential endogenous ligand that binds both isoforms equally might exist.

Equally important and exciting is the potential for this drug, already found to be safe in humans, to have multiple mechanisms that could be beneficial for treatment of diseases not yet considered. Two areas where we speculate that there could be potential are in autoimmunity and transplant rejection. While angiogenesis, stimulated by VEGF and other factors, can have a protective and regenerative role in response to tissue injury, it has also been linked to chronic inflammation, fibrosis, and tissue injury in both preclinical models and in human autoimmune diseases, including systemic lupus erythematosus, rheumatoid arthritis, vasculitis, and multiple sclerosis, to name a few[50]. Additionally, VEGF may play a role in acute and chronic rejection, with copious amounts of this growth factor released by immune cells leading over time to fibrosis and ultimately organ failure[51].

These data have made VEGF and its receptors an enticing target for future intervention in these disease processes. At the same time, we have already discussed a role for the AHR in the pathogenesis of both autoimmunity and organ rejection. We have a recent publication where ligands of the AHR can both inhibit, or alternatively accelerate rejection of skin grafts in fully mismatched mice, depending on the ligand utilized. Another study shows the ability of a ligand to promote tolerance to islet cell transplantation across a full MHC mismatch in mice[52]. These data would support the efficacy of a drug with these properties for treatment of autoimmunity and transplant rejection. There are already a few approved pharmaceuticals that likely function via the AHR (including treatments for asthma and organ rejection)[53], but none that combines the effect of VEGF blockade with modulation of the AHR. This could represent a novel angle to improve understanding of the mechanisms behind autoimmunity and organ rejection, and will provide a new class of drugs to combat these debilitating diseases.

Regarding previous in vitro and in vivo studies, there is strong data supporting a role for VEGF in immune cell migration and chemotaxis, generation of inflammatory cytokines, and angiogenesis. With that said, there are numerous studies that utilize SU5416 in experimental models and interpret the results based on its VEGF effect.

For example, one recent paper analyzed the role of VEGF in airway inflammation in vitro and in a murine model[33]. The inventors observation that SU5416 blocked LPS-induced airway inflammation, and specifically the differentiation of T cells to Th17 cells, along with a reduction of IL-6 would be fully consistent with regulatory effects of the drug through the AHR (this exact effect has been shown to be AHR-dependent when driven by TCDD and kynurenine[6,25]). While VEGF may also have a role in this differentiation, these data need to be interpreted carefully. In another study, daily injection of SU5416 is found to abrogate EAE in comparison to standard EAE induction with MOG peptide, which is presumed to be due to disruption of the effects of VEGF in this model[34]. Again, while it is possible that VEGF plays a role in EAE, these findings are identical to the results exhibited when animals in this protocol were treated with TCDD[6,7], which is AHR-dependent. Other studies have similarly used SU5416 to demonstrate the importance of VEGF in cell trafficking[35], although there does appear to be a role for VEGF in this mechanism shown with experiments that didn't involve SU5416. These are only a few of the hundreds of studies utilizing SU5416 to assess the importance of VEGF in various biologic mechanisms, as this has become a standard technique in experimental studies. While we are not asserting that VEGF is not involved in any of the above findings, consideration for a role of the AHR needs to be given.

Since it was reported that some ligands of the AHR favor Treg generation and others favor Th17 differentiation, we have been categorizing novel ligands for their properties in T-cell differentiation. The above data support that SU5416 enhances Treg generation in vitro, and that IDO is generated in pDCs in response to SU5416 in vitro in an AHR-dependent manner. We continue to characterize these effects for multiple ligands, and are considering theories explaining these differences including the potency and duration of binding of the ligands to the receptor, a possible change in conformation of the receptor when different ligands bind, and a possible effect on APC-T-cell interactions. That being said, there is some data to suggest that these dichotomous findings are not as clear cut as originally thought. Most of the in vitro studies examining effects on T-cell differentiation are done either in Treg or Th17 conditions, which are artificial by design.

In addition it has been shown that FICZ, the ligand best associated with Th17 differentiation, can enhance Treg differentiation in the presence of TGF-β, and TCDD can enhance Th17 differentiation[42,43]. This is similar to the data we show in FIG. 10, where SU5416 increases IL-17 in the supernatant of T cells cultured in Th17 conditions at low doses. It is likely that these effects are highly dependent on the ligand, the inflammatory milieu that is present in the assay or disease process, and the particular in vivo model system being studied. The prototypical regulatory ligand is TCDD, although others have been identified (kynurenine[25], ITE[44], VAF347[8]). FICZ remains the most well characterized effector ligand. By further delineating the properties of these ligands and the inflammatory milieu that allow them to have disparate effects on T-cell differentiation, it may ultimately be possible to utilize these properties to treat various diseases. This will require more characterization in vitro and in vivo. We do not believe the ligand activity is attributed to an indirect effect driven by VEGF, due to the impressive and rapid competitive binding in the radioligand assay, and additionally because we did test other known inhibitors of VEGFR-2, and did not find consistent DRE-luciferase activity in the range of their activity with VEGFR-2 (VegFR-2 IC50 values were in the nanomolar range, while AHR activity was in the micromolar range or not active) (FIG. 13). In addition to and independent of its effect on the AHR, SU5416 is certainly an inhibitor of VEGFR-2, as was well proven in previous studies[45]. The implications of our findings are important both for potential utility of this drug in humans, but also for mechanistic interpretations of previous experiments in vitro and in vivo.

### Methods

The KBA Library.

was screened at 10 μM per compound, by the Small Molecular Screening Facility of The Carbone Cancer Center of the University of Wisconsin School of Medicine and Public Health. This library represents the sum of three commercially available well characterized chemical libraries with a high frequency of approved drugs and prototype signaling molecules. This includes 2,000 diverse FDA approved drugs and natural products (Microsource Discovery Systems, Inc; Gaylordsville, Conn.); the 1280 compound LOPAC¹²⁸⁰ library of diverse characterized compounds (Sigma; St Louis, Mo.); and 880 characterized compounds (Prestwick Chemicals; Illkirch, FR).

The 2,000 compounds of the FDA approved drugs and natural products include: anthothecol, bussein, carapin, cedrelone, deacetylgedunin, 3-deacetylkhivorin, 7-deacetylkhivorin, dihydrogedunin, fissinolide, gedunin, deacetoxy-7-oxogedunin, 7-deacetoxy-7-oxokhivorin, khayanthone, 6-hydroxyangolensic acid methyl ester, mexicanolide, utilin, 3-deoxy-3beta-hydroxyangolensic acid methyl ester, prenyletin, carapin-8(9)-ene, 8beta-hydroxycarapin, 3,8-hemiacetal, hydrolysis product of bussein, 8-hydroxycarapinic acid, deoxykhivorin, 7-desacetoxy-6,7-dehydrogedunin, khivorin, epoxygedunin, 3-deoxo-3beta-acetoxydeoxydihydrogedunin, 3-alpha-acetoxydihydrodeoxygedunin, 3-deoxo-3beta-hydroxymexicanolide 16-enol ether, 1,2 alpha-epoxydeacetoxydihydrogedunin, podototarin, Totarol, strophanthidin, smilagenin acetate, hecogenin acetate, tigogenin, diosgenin, digitonin, Picrotin, 1,3-dideacetyl-7-deacetoxy-7-oxokhivorin, 1, 7-dideacetoxy-1,7-dioxo-3-deacetylkhivorin, 3,16-dideoxymexicanolide-3beta-diol, beta-amyrin, alpha-dihydrogedunol, xylocarpus a, deoxygedunin, deacetoxy(7)-7-oxokhivorinic acid, merogedunin, dihydrofissinolide, 3beta-acetoxydeoxodihydrogedunin, ptaeroxylin, entandrophragmin, peucenin, heteropeucenin, methyl ether, solasodine, obliquin, dictamnine, oleanoic acid, friedelin, beta-amyrin acetate, oxonitine, fraxidin methyl ether, tridesacetoxykhivorin, euphol acetate, gitoxigenin diacetate, xanthyletin, totarol-19-carboxylic acid, methyl ester, chukrasin methyl ether, angolensin (r), 3-alpha-hydroxy-4,4-bisnor-8,11,13-podocarpatriene, dihydrogedunic acid, methyl ester, homopterocarpin, strophanthidinic acid lactone acetate, formononetin, ichthynone, 3beta-hydroxydeoxodihydrodeoxygedunin, oleanolic acid acetate, imperatorin, smilagenin, beta-sitosterol, sitosteryl acetate, 5 alpha-androstan-3,17-dione, androsterone acetate, orsellinic acid, ethyl ester, 12a-hydroxy-9-demethylmunduserone-8-carboxylic acid, gambogic acid, haematoxylin, mundulone, brazilin, rotenone, isorotenone, leoidin, atranorin, gangaleoidin, griseofulvin, fumarprotocetraric acid, lecanoric acid, obtusaquinone, antiarol, theaflavin, pectolinarin, isotectorigenin, 7-methyl ether, asarylaldehyde, griseofulvic acid, 2′,4′-dihydroxychalcone 4′-glucoside, salsalate, flavanone, 4′-hydroxychalcone, 2,3,4′-trihydroxy-4-methoxybenzophenone, 2,6-dimethoxyquinone, koparin, 2,3,4-trihydroxy-4′-ethoxybenzophenone, 2,3-dihydroxy-4-methoxy-4′-ethoxybenzophenone, piscidic acid, roccellic acid, xanthoxylin, methylxanthoxylin, anhydrobrazilic acid, brazilein, deoxysappanone b 7,4′-dimethyl ether, 3-methylorsellinic acid, 2-benzoyl-5-methoxybenzoquinone, benzylhydrazine hydrochloride, ergosterol, ergosterol acetate, iretol, irigenin, iridin, dalbergione, 4-methoxy-4′-hydroxy-acacetin diacetate, apigenin, deoxysappanone b 7,3′-dimethyl ether, irigenin trimethyl ether, violastyrene, 4-methoxydalbergione, sappanone a dimethyl ether, deguelin(−), dehydrorotenone, duartin (−), irigenin, dibenzyl ether, irigenol, nonic acid, dalbergione, 7-hydroxy-2′-methoxyisoflavone, 2′-methoxyformonetin, deoxysappanone b trimethyl ether, deoxysappanone b 7,3′-dimethyl ether acetate, duartin, dimethyl ether, 4,4′-dimethoxydalbergione, dihydrorotenone, mundoserone, dehydrodihydrorotenone, 7-deshydroxypyrogallin-4-carboxylic acid, epigallocatechin 3,5-digallate, theaflavin digallate, garcinolic acid, tetrahydrogambogic acid, pomiferin, osajin, pachyrrhizin, pyrromycin, bisanhydrorutilantinone, rutilantinone, celastrol, lanosterol acetate, euphol, perseitol, epiafzelechin (2r,3r)(−), juarezic acid, haematommic acid, ethyl ester, epigallocatechin, avocatin a, phenylacetohydroxamic acid, citrinin, dihydromunduletone, cianidanol, epicatechin, epiafzelechin trimethyl ether, catechin tetramethylether, epicatechin monogallate, epigallocatechin-3-monogallate, theaflavin monogallates, genistein, gallic acid, tyramine, actinonin, purpurogallin, pyrogallin, dimethylcaffeic acid, dehydrovariabilin, chlorogenic acid, alizarin, khellin, iriginol hexaaceatate, 2-methoxyresorcinol, 7-methoxychromone, diphenylurea, metacetamol, alpha-toxicarol (dl), gentisic acid, methoxycarbonylsalicylic acid, hydroxyamphetamine hydrobromide, danthron, 4′-methoxychalcone, arabitol(d), cosmosiin, mequinol, hydroxytoluic acid, ononetin, veratric acid, chlorquinaldol, 1,3,5-trimethoxybenzene, peonol methyl ether, umbelliferone, 2-acetylpyrrole, 3,4-didesmethyl-5-deshydroxy-3′-ethoxyscleroin, perseitol heptaacetate, lanosterol, avocadene acetate, rotenonic acid, methyl ether, retusin 7-methyl ether, robustic acid, purpurogallin-4-carboxylic acid, 3,4-dimethoxydalbergione, pseudo-anisatin, avocadyne acetate, avocadene, arthonioic acid, haematoxylin pentaacetate, prasterone acetate, 3beta-hydroxy-23,24-bisnorchol-5-enic acid, alpha-hydroxydeoxycholic acid, methyl deoxycholate, stigmasta-4,22-dien-3-one, 5alpha-cholestanol, cholestan-3-one, cholestan-3b eta, 5 alpha,6beta-triol, cearoin, sinapic acid methyl ether, orsellinic acid, solidagenone, sphondin, stictic acid, euparin, agelasine (stereochemistry of diterpene unknown), isopimpinellin, pimpinellin, cholestane, lobaric acid, 1-monopalmitin, gibberellic acid, hydrocortisone, desoxycorticosterone acetate, isobergaptene, testosterone propionate, juglone, anisodamine hydrobromide, kobusone, 5-hydroxyiminoisocaryophyllene, senecrassidiol 6-acetate, bromo-3-hydroxy-4-(succin-2-yl)-caryolane gamma-lactone, cadin-4-en-10-ol, epi(13)torulosol, eupatoriochromene, metameconine, epoxy (1,11)humulene, 2-methylene-5-(2,5-dioxotetrahydrofuran-3-yl)-6-oxo-10,10-dimethylbicyclo[7:2:0]undecane, beta-caryophyllene alcohol, 3-amino-beta-pinene, 3-nor-3-oxopanasinsan-6-ol, 2-methoxy-5 (6)epoxy-tetrahydrocaryophyllene, isokobusone, 3,7-epoxycaryophyllan-6-ol, 2-hydroxy-5 (6)epoxy-tetrahydrocaryophyllene, 3,7-epoxycaryophyllan-6-one, clovanediol diacetate, vulpinic acid, usnic acid, eugenyl benzoate, 3-pinanone oxime, 15-norcaryophyllen-3-one, isopilocarpine nitrate, 5,7,4′-trimethoxyflavone, difucol hexamethyl ether, melezitose, andrographolide, isosafrole, isoreserpine, xanthopterin, arbutin, hymecromone methyl ether, santonin, chrysophanol, bergapten, phloridzin, sparteine sulfate, acetyl isogambogic acid, pelletierine hydrochloride, phloretin, gedunol, trichlormethine, menthone, metergoline, chrysanthemyl alcohol, phloracetophenone, rutoside (rutin), acetosyringone, salicylanilide, phenyl aminosalicylate, phenylbutyric acid, testosterone, 3-hydroxy-4-(succin-2-yl)-caryolane delta-lactone, dehydro (11,12)ursolic acid lactone, 3-oxoursan (28-13)olide, dehydroabietamide, muurolladie-3-one, bisabolol, bisabolol acetate, cedrol, d-limonene, aconitic acid, adonitol, anabasine hydrochloride, hydrastinine hydrochloride, djenkolic acid, epiandrosterone, helenine, hesperidin, hesperetin, cedryl acetate, chaulmoogric acid, chaulmoogric acid, ethyl ester, quinic acid, chrysanthemic acid, ethyl ester, phytol, hypoxanthine, larixinic acid, quassin, rhodinyl acetate, sanguinarine sulfate, alpha-tochopherol, alpha-tochopheryl acetate, visnagin, quinine ethyl carbonate, glucosaminic acid, thymoquinone, veratrine sulfate, dactinomycin, mitomycin c, coumophos, dimpylate, dichlorvos, malathion, chlorpyrifos, temefos, fenthion, propoxur, pyrethrins, lindane, mitotane, dibutyl phthalate, apiin, apiole, cycloveratrylene, piperonylic acid, d,l-threo-3-hydroxyaspartic acid, phenacylamine hydrochloride, haematoporphyrin, arginine hydrochloride, edetate disodium, evans blue, oxiglutatione disodium salt, guanidine hydrochloride, ivermectin, mannitol, meglumine, omega-3-acid esters (epa shown), sodium nitroprusside, sodium oxybate, sodium phenylacetate, sodium phenylbutyrate, sodium tetradecyl sulfate, sorbitol, adelmidrol, captamine, cyclamic acid, dehydroacetic acid, amylene hydrate, editol, d-(+)-maltose, meso-erythritol, lacitol, ipriflavone, haematommic acid, n-methylisoleucine, ethyl paraben, isopeonol, 2-hydroxy-3,4-dimethoxybenzoic acid, coumarin, n-methylbenzylamine hydrochloride, methoxyvone, sinapic acid, methyl robustone, cresopirine, derrustone, acetaminophen, acetazolamide, acetohydroxamic acid, acetylcholine chloride, acetylcysteine, adenosine, allopurinol, alverine citrate, amantadine hydrochloride, amikacin sulfate, amiloride hydrochloride, potassium p-aminobenzoate, aminocaproic acid, aminoglutethimide, aminosalicylate sodium, amitriptyline hydrochloride, amodiaquine dihydrochloride, amoxicillin, amphotericin b, ampicillin sodium, amprolium, antazoline phosphate, anthralin, antipyrine, apomorphine hydrochloride, aspirin, atropine sulfate, aurothioglucose, azathioprine, bacitracin, baclofen, beclomethasone dipropionate, benserazide hydrochloride, benzethonium chloride, benzocaine, benzthiazide, beta-carotene, betamethasone, betamethasone valerate, bethanechol chloride, bisacodyl, bithionate sodium, bromocriptine mesylate, busulfan, caffeine, camphor (1r), capreomycin sulfate, carbachol, carbamazepine, carbenicillin di sodium, carbinoxamine maleate, carisoprodol, cefadroxil, cefazolin sodium, cefotaxime sodium, cephalothin sodium, cephapirin sodium, cephradine, cetylpyridinium chloride, chlorambucil, chloramphenicol palmitate, chloramphenicol sodium succinate, chloramphenicol, chlorcyclizine hydrochloride, chlorhexidine hydrochloride, chlorocresol, chloroquine diphosphate, chlorothiazide, chloroxylenol, chlorpheniramine (s) maleate, chlorpromazine, chlorpropamide, chlortetracycline hydrochloride, chlorthalidone, chlorzoxazone, ciclopirox olamine, cinoxacin, clemastine fumarate, clidinium bromide, clindamycin hydrochloride, clofibric acid, clomiphene citrate, clonidine hydrochloride, clotrimazole, cloxacillin sodium, cloxyquin, colchicine, colistimethate sodium, cortisone acetate, cotinine, cresol, cromolyn sodium, cyclizine, cyclopentolate hydrochloride, cyclophosphamide hydrate, cycloserine (d), cyproterone acetate, cytarabine, dacarbazine, danazol, dapsone, daunorubicin, deferoxamine mesylate, sodium dehydrocholate, demeclocycline hydrochloride, desipramine hydrochloride, dexamethasone, dexamethasone acetate, dexamethasone sodium phosphate, dextromethorphan hydrobromide, dibenzothiophene, dibucaine hydrochloride, diclofenac sodium, dicloxacillin sodium, dicumarol, dicyclomine hydrochloride, dienestrol, diethylcarbamazine citrate, diethylstilbestrol, diflunisal, digitoxin, digoxin, dihydroergotamine mesylate, dihydrostreptomycin sulfate, dimenhydrinate, dimercaprol, dimethadione, dioxybenzone, diphenhydramine hydrochloride, diphenylpyraline hydrochloride, dipyridamole, pyrithione zinc, disopyramide phosphate, disulfiram, dopamine hydrochloride, doxepin hydrochloride, doxycycline hydrochloride, doxylamine succinate, dyclonine hydrochloride, dyphylline, trisodium ethylenediamine tetracetate, emetine, ephedrine (1r,2s) hydrochloride, epinephrine bitartrate, equilin, ergocalciferol, ergonovine maleate, erythromycin ethylsuccinate, erythromycin, erythromycin stearate, estradiol, estradiol cypionate, estradiol valerate, estriol, estrone, ethacrynic acid, ethambutol hydrochloride, ethinyl estradiol, ethionamide, ethopropazine hydrochloride, eucalyptol, eucatropine hydrochloride, eugenol, fludrocortisone acetate, flumethazone pivalate, fluocinolone acetonide, fluocinonide, fluorometholone, fluorouracil, flurbiprofen, furazolidone, furosemide, fusidic acid, gallamine triethiodide, gemfibrozil, gentamicin sulfate, gentian violet, glucosamine hydrochloride, gramicidin, guaifenesin, guanabenz acetate, guanethidine sulfate, halazone, haloperidol, hetacillin potassium, hexachlorophene, hexylresorcinol, histamine dihydrochloride, homatropine bromide, homatropine methylbromide, hydralazine hydrochloride, hydrochlorothiazide, hydrocortisone acetate, hydrocortisone hemisuccinate, hydrocortisone phosphate triethylamine, hydroflumethiazide, hydroxyprogesterone caproate, hydroxyurea, hydroxyzine pamoate, hyoscyamine, ibuprofen, imipramine hydrochloride, indapamide, indomethacin, indoprofen, inositol, iodoquinol, ipratropium bromide, isoniazid, isopropamide iodide, isoproterenol hydrochloride, isosorbide dinitrate, isoxsuprine hydrochloride, kanamycin a sulfate, ketoconazole, lactulose, leucovorin calcium, levonordefrin, lincomycin hydrochloride, mafenide hydrochloride, maprotiline hydrochloride, mecamylamine hydrochloride, mechlorethamine, meclizine hydrochloride, meclofenamate sodium, medroxyprogesterone acetate, medrysone, megestrol acetate, melphalan, mepenzolate bromide, mercaptopurine, mestranol, metaproterenol, methacholine chloride, methenamine, methicillin sodium, methimazole, methocarbamol, methotrexate(+/−), methoxamine hydrochloride, methoxsalen, methscopolamine bromide, methyldopa, methylergonovine maleate, methylprednisolone, methylthiouracil, metoclopramide hydrochloride, metoprolol tartrate, metronidazole, miconazole nitrate, minocycline hydrochloride, minoxidil, moxalactam disodium, nadide, nafcillin sodium, naloxone hydrochloride, naphazoline hydrochloride, naproxen(+), neomycin sulfate, neostigmine bromide, niacin, nifedipine, nitrofurantoin, nitrofurazone, nitromide, norepinephrine, norethindrone, norethindrone acetate, norethynodrel, norfloxacin, norgestrel, nortriptyline, noscapine hydrochloride, novobiocin sodium, nylidrin hydrochloride, nystatin, orphenadrine citrate, oxacillin sodium, oxidopamine hydrochloride, oxybenzone, oxymetazoline hydrochloride, oxyphenbutazone, oxyquinoline hemisulfate, oxytetracycline, papaverine hydrochloride, parachlorophenol, pargyline hydrochloride, penicillamine, penicillin g potassium, penicillin v potassium, phenacemide, phenazopyridine hydrochloride, phenelzine sulfate, phenindione, pheniramine maleate, phenolphthalein, phenylbutazone, phenylephrine hydrochloride, phenylpropanolamine hydrochloride, phenytoin sodium, physostigmine salicylate, pilocarpine nitrate, pindolol, piperacillin sodium, piperazine, piroxicam, polymyxin b sulfate, praziquantel, prazosin hydrochloride, prednisolone, prednisolone acetate, prednisone, primaquine diphosphate, primidone, probenecid, procainamide hydrochloride, procaine hydrochloride, prochlorperazine edisylate, procyclidine hydrochloride, progesterone, promazine hydrochloride, promethazine hydrochloride, propantheline bromide, dexpropranolol hydrochloride, propylthiouracil, pseudoephedrine hydrochloride, pyrantel pamoate, pyrazinamide, pyrilamine maleate, pyrimethamine, pyrvinium pamoate, quinacrine hydrochloride, quinidine gluconate, quinine sulfate, racephedrine hydrochloride, reserpine, resorcinol, rifampin, roxarsone, salicyl alcohol, salicylamide, sodium salicylate, scopolamine hydrobromide, sisomicin sulfate, spectinomycin hydrochloride, spironolactone, streptomycin sulfate, streptozosin, sulfabenzamide, sulfacetamide, sulfadiazine, sulfamerazine, sulfamethazine, sulfamethizole, sulfamethoxazole, sulfapyridine, sulfasalazine, sulfathiazole, sulfinpyrazone, sulfisoxazole, sulindac, tamoxifen citrate, terbutaline hemisulfate, tetracaine hydrochloride, tetracycline hydrochloride, tetrahydrozoline hydrochloride, theophylline, thiabendazole, thimerosal, thioguanine, thioridazine hydrochloride, thiothixene, timolol maleate, tobramycin, tolazoline hydrochloride, tolbutamide, tolmetin sodium, tolnaftate, tranylcypromine sulfate, triacetin, triamcinolone, triamcinolone acetonide, triamcinolone diacetate, triamterene, trichlormethiazide, trifluoperazine hydrochloride, trihexyphenidyl hydrochloride, trimeprazine tartrate, trimethobenzamide hydrochloride, trimethoprim, trioxsalen, tripelennamine citrate, triprolidine hydrochloride, tropicamide, tryptophan, tuaminoheptane sulfate, tubocurarine chloride, tyrothricin, urea, ursodiol, valproate sodium, vancomycin hydrochloride, vidarabine, vinblastine sulfate, warfarin, xylometazoline hydrochloride, zomepirac sodium, acetarsol, acriflavinium hydrochloride, aminopyrine, broxyquinoline, carnitine (dl) hydrochloride, dichlorophene, flopropione, hexestrol, hexetidine, iproniazid sulfate, mecysteine hydrochloride, merbromin, octopamine hydrochloride, pentamidine isethionate, phenacetin, phenylmercuric acetate, pregnenolone, sulfanilamide, vincamine, azelaic acid, theobromine, strychnine, aconitine, ajmaline, hydroquinidine, yohimbine hydrochloride, acebutolol hydrochloride, acemetacin, adenosine phosphate, ketotifen fumarate, benfluorex hydrochloride, betahistine hydrochloride, dihydrofolic acid, quercetin, molsidomine, mycophenolic acid, oleandomycin phosphate, ouabain, albuterol (+/−), gamma-aminobutyric acid, aminopterin, arecoline hydrobromide, captopril, cimetidine, clozapine, hydrastine (1r, 9s), kynurenic acid, lidocaine hydrochloride, melatonin, phentolamine hydrochloride, acetyltryptophanamide, acetyltryptophan, acetylglutamic acid, n-acetylproline, citropten, chrysin, acetylglucosamine, 6,4′-dihydroxyflavone, 7,2′-dihydroxyflavone, 5,7-dihydroxy-4-methylcoumarin, 4-methylesculetin, 3,4′-dimethoxyflavone, 3,6-dimethoxyflavone, 3,7-dimethoxyflavone, chrysin dimethyl ether, naringenin, neohesperidin dihydrochalcone, quercitrin, xanthurenic acid, nalidixic acid, lobeline hydrochloride, quinalizarin, hecogenin, guanidine carbonate, rosolic acid, calcein, kinetin, naringin, butamben, cefaclor, iodipamide, levothyroxine, liothyronine, allantoin, alloxan, althiazide, adenine, aminacrine, berberine chloride, bekanamycin sulfate, budesonide, betulin, carminic acid, bergenin, bicuculline (+), brucine, canrenoic acid, potassium salt, carylophyllene oxide, ursocholanic acid, cephalosporin c sodium, chenodiol, cholecalciferol, cinchonidine, cholic acid, cinchonine, caryophyllene [t(−)], lathosterol, coenzyme b12, cholesterol, cholest-5-en-3-one, citrulline, bilirubin, s-isocorydine (+), coralyne chloride, boldine, harmalol hydrochloride, harmane, harmine, mimosine, norharman, palmatine chloride, piperine, corynanthine, kynurenine, trigonelline, emodin, esculetin, aesculin, etoposide, cholic acid, methyl ester, lithocholic acid, dehydrocholic acid, protoveratrine a, carnosine, gitoxin, glutamine (d), 18alpha-glycyrrhetinic acid, enoxolone, glycyrrhizic acid, ammonium salt, flumequine, flunarizine hydrochloride, fluphenazine hydrochloride, flutamide, glafenine, ethoxyquin, drofenine hydrochloride, ethaverine hydrochloride, droperidol, famotidine, dropropizine, etodolac, fenoterol hydrobromide, fenbufen, 1r,9s-hydrastine, fenofibrate, fenoprofen, 3-hydroxyflavone, flufenamic acid, fenbendazole, ferulic acid, berbamine hydrochloride, 6-hydroxyflavone, fenspiride hydrochloride, fendiline hydrochloride, mefenamic acid, methacycline hydrochloride, puromycin hydrochloride, picrotoxinin, mefexamide, probucol, mebendazole, protoporphyrin ix, pimethixene maleate, nalbuphine hydrochloride, mebhydrolin naphthalenesulfonate, mebeverine hydrochloride, meclocycline sulfosalicylate, proglumide, minaprine hydrochloride, memantine hydrochloride, aceclidine, tramiprosate, 5-aminopentanoic acid hydrochloride, atenolol, capsaicin, carbetapentane citrate, fampridine, nerol, nicergoline, pimozide, nicardipine hydrochloride, nefopam, pirenzepine hydrochloride, pramoxine hydrochloride, mephenesin, sulfachlorpyridazine, sulfaphenazole, sulfadimethoxine, sulfaquinoxaline sodium, sulfaguanidine, sulfamonomethoxine, sulconazole nitrate, ritodrine hydrochloride, sulpiride, ranitidine, spiperone, suloctidil, ronidazole, sulfameter, sulfamethoxypyridazine, suxibuzone, sulmazole, suprofen, acetaminosalol, saccharin, azobenzene, acetanilide, todralazine hydrochloride, flurandrenolide, erythromycin estolate, estradiol benzoate, econazole nitrate, flunisolide, ebselen, tolperisone hydrochloride, flumethasone, tolfenamic acid, zaprinast, xylazine, tolazamide, galanthamine, retinol, lanatoside c, kinetin riboside, karanjin, humulene (alpha), mucic acid, linalool (+), enalapril maleate, ketoprofen, lisinopril, acetyl-1-leucine, bufexamac, bumetanide, carbenoxolone sodium, carprofen, ciprofibrate, diacerin, fosfosal, isotretinon, mesna, niflumic acid, tretinoin, bretylium tosylate, foscarnet sodium, folic acid, phthalylsulfathiazole, pipemidic acid, succinylsulfathiazole, tranexamic acid, cephalexin, cefsulodin sodium, oxolinic acid, cefoxitin sodium, suramin, cefuroxime sodium, metampicillin sodium, lomefloxacin hydrochloride, cefamandole sodium, fosfomycin calcium, cefmetazole sodium, cefamandole nafate, cefoperazone, pralidoxime mesylate, ofloxacin, piromidic acid, bezafibrate, liothyronine (1-isomer) sodium, alrestatin, 5-chloroindole-2-carboxylic acid, 6,7-dichloro-3-hydroxy-2-quinoxalinecarboxylic acid, imidazol-4-ylacetic acid sodium salt, 4-naphthalimidobutyric acid, indole-2-carboxylic acid, n-(9-fluorenylmethoxycarbonyl)-1-leucine, proadifen hydrochloride, cyclocreatine, 5-fluoroindole-2-carboxylic acid, alpha-cyano-3-hydroxycinnamic acid, alpha-cyano-4-hydroxycinnamic acid, perillic acid (−), quinolinic acid, carboplatin, cisplatin, antimycin a (a1 shown), zidovudine [azt], azacitidine, cycloheximide, azaserine, p-fluorophenylalanine, tinidazole, cycloleucine, aminocyclopropanecarboxylic acid, carbidopa, p-chlorophenylalanine, piracetam, ethosuximide, piperidolate hydrochloride, anisindione, cyclosporine, troleandomycin, 1-leucyl-1-alanine, phenethyl caffeate (cape), tilarginine hydrochloride, resveratrol, cadaverine tartrate, abietic acid, ascorbic acid, cellobiose (d[+]), camptothecin, abscisic acid (cis,trans; +/−), harmol hydrochloride, amygdalin, ellagic acid, glutathione, guaiazulene, lawsone, nerolidol, monocrotaline, hematein, menadione, salicin, monensin sodium (monensin a is shown), morin, abamectin (avermectin b1a shown), quebrachitol, dilazep dihydrochloride, flurofamide, levetiracetam, melperone hydrochloride, vorinostat, clofarabine, capecitabine, vinorelbine, docetaxel, gefitinib, dasatinib, imatinib, tandutinib, anastrozole, fulvestrant, oxaliplatin, dimesna, amisulpride, nelarabin, candesartan, temozolamide, diaveridine, lomustine, lornoxicam, lomerizine hydrochloride, iohexol, meropenem, moguisteine, nadifloxacin, nefazodone hydrochloride, nefiracetam, nifursol, oltipraz, paliperidone, pazufloxacin mesylate, penciclovir, pidotimod, piceid, pranoprofen, prasugrel, procarbazine hydrochloride, prulifloxacin, pyronaridine tetraphosphate, quinestrol, racecadotril, ractopamine hydrochloride, rasagiline, roxatidine acetate hydrochloride, rufloxacin hydrochloride, seratrodast, sparfloxacin, stavudine, sulbactam, tazobactam, tenatoprazole, tibolone, tropisetron hydrochloride, uracil, voriconazole, avermectin ala, diclazuril, diminazene aceturate, idoxurdine, imiquimod, irsogladine maleate, itopride hydrochloride, letrozole, levonorgestrel, levosimendan, lofexidine hydrochloride, doxofylline, difloxacin hydrochloride, zalcitabine, esomeprazole potassium, ganciclovir, dexibuprofen, idoxuridine, granisetron hydrochloride, fasudil hydrochloride, sodium cyclamate, aceglutamide, cryoflurane, amorolfine hydrochloride, cyacetacide, ramoplanin [a2 shown; 2 mm], pitavastatin calcium, aceclofenac, adapalene, amlexanox, daptomycin, deflazacort, entacapone, eprinomectin, erdosteine, estramustine, linezolid, loxoprofen, lumiracoxib, manidipine hydrochloride, olsalazine sodium, pemetrexed, ritonavir, spirapril hydrochloride, tenofovir, tigecycline, alprazolam, estradiol dipropionate, abacavir sulfate, acenocoumarol, acedoben, actarit, balsalazide disodium, brinzolamide, cefepime hydrochloride, cefmenoxime hydrochloride, ceforanide, cefotetan, saxagliptin, ondansetron, bornyl acetate, chlorobutanol, cintriamide, benzyl alcohol, benzoic acid, benzyl benzoate, benzoyl peroxide, benzyl isothiocyanate, betaine hydrochloride, biotin, aklomide, aminothiazole, monobenzone, dipyrocetyl, dinitolmide, nicotinyl alcohol tartrate, aminohydroxybutyric acid, artemisinin, bucladesine, chromocarb, acexamic acid, retinyl acetate, pipenzolate bromide, floxuridine, altretamine, aminohippuric acid, mefloquine, adiphenine hydrochloride, alexidine hydrochloride, quinapril hydrochloride, pridinol methanesulfonate, aniracetam, ambroxol hydrochloride, amifostine, inamrinone, pyrithyldione, tiapride hydrochloride, gluconolactone, tioxolone, urapidil hydrochloride, azlocillin sodium, bacampicillin hydrochloride, bendroflumethiazide, benfotiamine, bepridil hydrochloride, bromhexine hydrochloride, bromopride, carmustine, ceftriaxone sodium trihydrate, dibekacin, vinpocetine, trimipramine maleate, triflupromazine hydrochloride, trazodone hydrochloride, dequalinium chloride, levomenthol, thonzylamine hydrochloride, thiamphenicol, tenoxicam, cetrimonium bromide, chloroxine, chlorprothixene hydrochloride, cinnarazine, citiolone, clofoctol, cyclobenzaprine hydrochloride, dantrolene sodium, betamethasone 17,21-dipropionate, dobutamine hydrochloride, edoxudine, enoxacin, heptaminol hydrochloride, diosmin, ethisterone, fipexide hydrochloride, pararosaniline pamoate, lonidamine, meclofenoxate hydrochloride, perhexiline maleate, paromomycin sulfate, methapyrilene hydrochloride, nifenazone, nimesulide, propiolactone, halcinonide, hycanthone, pyridostigmine bromide, isoxicam, labetalol hydrochloride, levamisole hydrochloride, mephentermine sulfate, metaraminol bitartrate, methazolamide, methylbenzethonium chloride, methylprednisolone sodium succinate, amsacrine, midodrine hydrochloride, nadolol, naltrexone hydrochloride, cyclothiazide, niclosamide, nocodazole, nomifensine maleate, pergolide mesylate, prilocaine hydrochloride, hydrocortisone butyrate, roxithromycin, mitoxantrone hydrochloride, oxethazaine, hexamethonium bromide, dipyrone, sulfanilate zinc, urethane, timonacic, thiram, thiotepa, thiodiglycol, tetroquinone, sulfanitran, chloroacetoxyquinoline, oxibendazole, pasiniazid, pempidine tartrate, d-phenylalanine, pipobroman, exalamide, nafronyl oxalate, quipazine maleate, ritanserin, semustine, spiramycin, zopiclone, aloin, choline chloride, clofibrate, cytidine, mepartricin, resorcinol monoacetate, nimodipine, salinomycin, sodium, acyclovir, retinyl palmitate, cypermethrin, thalidomide, nitrendipine, benzalkonium chloride, pentoxifylline, ciprofloxacin, safrole, pronetalol hydrochloride, spaglumic acid, 3,5-dinitrocatechol (or-486), edaravone, n-methyl-d-aspartic acid (nmda), metitepine maleate, 1-phenylbiguanide hydrochloride, gaboxadol hydrochloride, nabumetone, sodium thioglycolate, celecoxib, azithromycin, sevoflurane, bleomycin (bleomycin b2 shown), anethole, terfenadine, clopidogrel sulfate, orbifloxacin, loratadine, selamectin, enrofloxacin, atorvastatin calcium, naproxol, azadirachtin, colforsin, desacetylcolforsin, homidium bromide, isosorbide mononitrate, amcinonide, bupivacaine hydrochloride, ramifenazone, drospirenone, ecamsule triethanolamine, enalaprilat, gadoteridol, iodixanol, iothalamic acid, ioversol, ioxilan, lamivudine, mangafodipir trisodium, methyclothiazide, nevirapine, nitazoxanide, terconazole, viomycin sulfate, ziprasidone mesylate, acetophenazine maleate, azatadine maleate, bemotrizinol, benzonatate, betazole hydrochloride, bisoctrizole, candicidin, carbarsone, cefpiramide, valganciclovir hydrochloride, travoprost, thonzonium bromide, piperacetazine, piroctone olamine, prednicarbate, quinethazone, tiletamine hydrochloride, albendazole, patulin, anisomycin, paclitaxel, butacaine, clenbuterol hydrochloride, clobetasol propionate, cloperastine hydrochloride, cyproterone, tryptamine, iopanic acid, ketorolac tromethamine, lansoprazole, leflunomide, mexiletine hydrochloride, morantel citrate, oxyphencyclimine hydrochloride, pentolinium tartrate, perphenazine, propafenone hydrochloride, periciazine, ribavirin, ribostamycin sulfate, spermidine trihydrochloride, thioctic acid, cacodylic acid, putrescine dihydrochloride, tulobuterol, tacrolimus, methoxy amine hydrochloride, 2-thiouracil, dimethyl 4,4-o-phenylene-bis (3-thiophanate), fluconazole, lovastatin, hydroxychloroquine sulfate, agmatine sulfate, batyl alcohol, anebrompheniramine maleate, caffeic acid, glucitol-4-gucopyanoside, conessine, convallatoxin, baicalein, centaurein, sirolimus, levulinic acid, 3-benzylidenyl-, ubidecareneone, alpha-mangostin, hederagenin, gossypol, evoxine, lupinine, salsolidine, thermopsine perchlorate, lagochilin, salsoline, cytisine, beta-escin, 3-deshydroxysappanol trimethyl ether, triacetylresveratrol, resveratrol 4′-methyl ether, stigmasterol, fucostanol, epicoprosterol, physcion, rhoifolin, yohimbic acid hydrate, sennoside a, tomatine, betulinic acid, paroxetine hydrochloride, ethylnorepinephrine hydrochloride, teniposide, phenothrin, sildenafil citrate, methoprene (s), tetrachloroisophthalonitrile, dihydrojasmonic acid, tannic acid, hieracin, formestane, diffractaic acid, linamarin, 3,7-dihydroxyflavone, 6,3′-dimethoxyflavone, trimedlure, alaproclate, ancitabine hydrochloride, arachidonic acid, butoconazole, acetriazoic acid, artenimol, dirithromycin, gliclazide, mepivacaine hydrochloride, meloxicam sodium, nimustine, nilutamide, norcantharidin, aphyllic acid, anabasamine hydrochloride, lomatin, selinidin, ursinoic acid, peucedanin, venlafaxine, citalopram hydrobromide, fluoxetine, bupropion, cefuroxime axetil, rhetsinine, fexofenadine hydrochloride, asarinin (−), trifluridine, aminolevulinic acid hydrochloride, tetrandrine, telenzepine hydrochloride, pirenperone, avobenzone, piperonyl butoxide, isoliquiritigenin, pramipexole dihydrochloride, canthaxanthin (euglenanone), bovinocidin (3-nitropropionic acid), diplosalsalate, atovaquone, chloroguanide hydrochloride, chloranil, n-acetylaspartic acid, trimetozine, zoxazolamine, acrisorcin, phenylbutyrate sodium, fenbutyramide, 2-mercaptobenzothiazole, cysteamine hydrochloride, crustecdysone, metaxalone, clarithromycin, 2,3-dihydroxy-6,7-dichloroquinoxaline, rofecoxib, simvastatin, hydroquinone, 1,4-naphthoquinone, oxcarbazepine, betamipron, epicatechin pentaacetate, carvedilol, nateglinide, irbesartan, levofloxacin, candesartan cilextil, rosiglitazone, losartan, lithium citrate, gatifloxacin, miglitol, desvenlafaxine succinate, dexlansoprazole, armodafinil, galangin, febuxostat, orlistat, valdecoxib, moxifloxacin hydrochloride, proflavine hemisulfate, pioglitazone hydrochloride, donepezil hydrochloride, ornithine aketoglutarate, picropodophyllin, picropodophyllin acetate, podophyllin acetate, bisorcic, beta-naphthol, bitoscanate, carsalam, carzenide, tosylchloramide sodium, chlorindione, cinchophen, benorilate, chiniofon, chloralose, deferiprone, decimemide, diacetamate, doxifluridine, efloxate, 7-hydroxyflavone, epiestriol, fluindarol, ftaxilide, ornithine, orotic acid, firocoxib, trilostane, mycophenolate mofetil, gyromitrin, formylmethylhydrazine, mepiroxol, isaxonine, beta-peltatin, chrysanthemic acid, milnacipran hydrochloride, retapamulin, ibandronate sodium, fast green fcf, bicalutamide, artesunate, ampiroxicam, acipimox, ceftiofur hydrochloride, climbazole, clinafoxacin hydrochloride, closantel, dihydrojasmonic acid, methyl ester, fluvastatin, olseltamivir phosphate, levocetirizine dihydrochloride, glimepiride, captan, galangin trimethyl ether, sclareolide, 3-hydroxyindole, 4-hydroxyindole, permethrin, puerarin, pizotyline malate, 1-deoxyalliin, 2,3-dimercaptosuccinic acid, trimebutine maleate, exemestane, astragaloside iv, homosalate, propofol, demethylnobiletin, artemether, artemotil, baccatin iii, tanshinone iia sulfonate sodium, kasugamycin hydrochloride, 2,5-di-t-butyl-4-hydroxyanisole, bisphenol a, salvinorin a, cyclandelate, prasterone, 1-buthionine sulfoximine, thiostrepton, tilmicosin, flunixin meglumine, clioquinol, clorsulon, estropipate, estragole, aminoethylisothiourea dihydrobromide, 11a-acetoxyprogesterone, aztreonam, 21-acetoxypregnenolone, clavulanate lithium, alclometazone dipropionate, dicyclohexylurea, gossypin, plumbagin, 3,4-dimethoxycinnamic acid, 2′,4′-dihydroxychalcone, mangiferin, piplartine, 2′,5′-dihydroxy-4-methoxychalcone, gossypetin, harpagoside, 2′,4′-dihydroxy-4-methoxychalcone, 2′,3-dihydroxy-4,4′,6′-trimethoxychalcone, 3-amino-1,2,4-triazole, 3-hydroxytyramine, n-phenylanthranilic acid, 2,2′-azo-bis-2-aminopropane, aurin tricarboxylic acid, 4,4′-diisothiocyanostilbene-2,2′-sufonic acid sodium salt, 2-methyl-4-(piperidin-1-ylcarboxy)-5-isopropylphenyltrimethylammonium chloride, alendronate sodium, acadesine, ethacridine lactate, desoxypeganine hydrochloride, triflumuron, diflubenzuron, acarbose, bambuterol hydrochloride, garlicin, asiatic acid, rubescensin a, ropinirole, 6,2′-dimethoxyflavone, quetiapine, rizatriptan benzoate, epitestosterone, benazepril hydrochloride, famciclovir, amlodipine besylate, ezetimibe, almotriptan, olmesartan medoxomil, olmesartan, ceftibuten, cefdinir, valsartan, sibutramine hydrochloride, perindopril erbumine, rosuvastatin calcium, ramipril, tegaserod maleate, escitalopram oxalate, rhodocladonic acid, baeomycesic acid, deracoxib, cilostazol, avocadyne, lupanyl acid hydrochloride, sparteine hydroiodide, lupanine perchlorate, citicoline, troxerutin, ginkgolic acid, canrenone, apramycin, madecassic acid, imidacloprid, palmatine, sinomenine, theanine, huperzine a, telmisartan, sertraline hydrochloride, alfluzosin, trandolapril, telithromycin, oxaprozin, nobiletin, tangeritin, propranolol hydrochloride (+/−), crotamiton, benzanthrone, capsanthin, frovatriptan succinate, zolmitriptan, nonoxynol-9, diallyl sulfide, carbadox, oxfendazole, perillyl alcohol, 3-isobutyl-1-methylxanthine (ibmx), amitraz, purpurin, geneticin, secnidazole, pefloxacine mesylate, aspartame, triadimefon, chlorophyllide cu complex na salt, bifonazole, rebamipide, dibenzoylmethane, tylosin tartrate, sarafloxacin hydrochloride, 6-aminonicotinamide, protionamide, carmofur, clopidol, indole-3-carbinol, rifaximin, cepharanthine, carbimazole, bissalicyl fumarate, solanesol, solanesyl acetate, chlormadinone acetate, 4′-demethylepipodophyllotoxin, miltefosine, oxiconazole nitrate, 3,3′-diindolylmethane, azaperone, tranilast, securinine, valeryl salycilate, elaidylphosphocholine, 1-phenylalaninol, azelastine hydrochloride, nomilin, 7-nitroindazole, curcumin, ketanserin tartrate, riboflavin, riluzole, 4-o-methylphloracetophenone, fipronil, lufenuron, decoquinate, nitenpyram, dimethylsulfone, cefditorin pivoxil, modafinil, hygromycin b, cefprozil, ranolazine, mometasone furoate, valacyclovir hydrochloride, zolpidem, cetirizine hydrochloride, sumatriptan succinate, vardenafil hydrochloride, acedapsone, 3,4′,5,6,7-pentamethoxyflavone, sinensetin, 5-hydroxy-2′,4′,7,8-tetramethoxyflavone, hexamethylquercetagetin, methyldopate hydrochloride, atomoxetine hydrochloride, dutasteride, duloxetine hydrochloride, tramadol hydrochloride, ni soldipine, montelukast sodium, terbinafine hydrochloride, desloratidine, moxidectin, bicuculline(−) methiodide, fluorescein, niacinamide, phenylethyl alcohol, oxybutynin chloride, benurestat, benzoxiquine, bismuth sub salicylate, benzoylpas, bromindione, capobenic acid, dexpanthenol, acetohexamide, ethoxzolamide, ethotoin, flucytosine, flurothyl, fomepizole hydrochloride, glipizide, halothane, guanfacine hydrochloride, d-lactitol monohydrate, levocarnitine, hydrocortisone valerate, lobendazole, methsuximide, methylene blue, methylatropine nitrate, naftifine hydrochloride, octodrine, nithiamide, pralidoxime chloride, prednisolone hemisuccinate, pyridoxine, quinaprilat, paramethadione, phensuccimide, rimantadine hydrochloride, sulfisoxazole acetyl, sulisobenzone, taurine, thiamine, triclosan, trimethadione, zinc undecylenate, undecylenic acid, clindamycin palmitate hydrochloride, cefonicid sodium, helicin, ifosfamide, beta-mangostin, netilmicin sulfate, doxorubicin, lupeol, phytonadione, lupeol acetate, salidroside, milrinone, apigenin dimethyl ether, methysergide maleate, solifenacin succinate, acepromazine maleate, mesoridazine besylate, benoxinate hydrochloride, betaxalol hydrochloride, biperiden, carteolol hydrochloride, dexchlorpheniramine maleate, diloxanide furoate, doxapram hydrochloride, dydrogesterone, etidronate disodium, fenoldipam mesylate, guanadrel sulfate, levobunolol hydrochloride, meprylcaine hydrochloride, natamycin, norgestimate, penbutolol sulfate, propoxycaine hydrochloride, terazosin hydrochloride, tioconazole, ergotamine tartrate, anagrelide hydrochloride, etomidate, felbamate, fenretinide, fluticasone propionate, fluvoxamine maleate, lamotrigine, mifepristone, raloxifene hydrochloride, cefpodoxime proxetil, tadalafil, aminopentamide sulfate, arsanilic acid, panthenol (dl), phentermine, vincristine sulfate, trientine hydrochloride, ticlopidine hydrochloride, ticarcillin disodium, tetramizole hydrochloride, toltrazuril, toremiphene citrate, rolipram, rolitetracycline, proparacaine hydrochloride, pipamperone, penfluridol, pancuronium bromide, omeprazole, flumazenil, altrenogest, bisoprolol fumarate, fludarabine phosphate, mupirocin, teicoplanin [a(2-1) shown], epirubicin hydrochloride, vecuronium bromide, aliskiren hemifumarate, acamprosate calcium, prednisolone sodium phosphate, pregnenolone succinate, darifenacin hydrobromide, pantothenic acid(d) na salt, algestone acetophenide, desoxymetasone, betamethasone acetate, isofluprednone acetate, betamethasone sodium phosphate, desonide, melengestrol acetate, phthalylsulfacetamide, docosanol, levocarnitine propionate hydrochloride, lipoamide, ascorbyl palmitate, aleuretic acid, androsterone, 7-aminocephalosporanic acid, borneol, chlorophyll, erythrosine sodium, glycopyrrolate, idebenone, itraconazole, melibiose, skatole, oxtriphylline, octisalate, oxythiamine chloride hydrochloride, pyritinol, riboflavin 5-phosphate sodium, spermine, trichlorfon, syringic acid, furaltadone, bucetin, famprofazone, oxelaidin citrate, agaric acid, diatrizoic acid, bephenium hydroxynapthoate, dichlorisone acetate, diperodon hydrochloride, selegiline hydrochloride, ceftazidime, oxyphenonium bromide, camylofine dihydrochloride, triclabendazole, nifuroxazide, erythrose, topiramate, gemifloxacin mesylate, pravastatin sodium, gabapentin, avocadanofuran, avocadenofuran, peoniflorin, levalbuterol hydrochloride, cryptotanshinone, metformin hydrochloride, pregabalin, pantoprazole, eletriptan hydrobromide, topotecan hydrochloride, irinotecan hydrochloride, tanshinone iia, dihydrotanshinone i, beclamide, strychnine methiodide, piperic acid, desloratadine hydrochloride, laccaic acid a, ethionine, ampyzine sulfate, aripiprazole, cresotamide, 2′,4′,6′-trimethoxyacetophenone, 2-aminobenzenesulfonamide, 3-acetamidocoumarin, 3-acetylcoumarin, ampyrone, 4-hydroxyantipyrine, atracurium besylate, chicago sky blue, diflorasone diacetate, felodipine, fusaric acid, glycocholic acid, gramine, hydroquinine hydrobromide hydrate, doramectin, cyromazine, colesevalam hydrochloride (high mol wt copolymer @10 mg/ml), zileuton, methylphenidate hydrochloride, mangostin trimethyl ether, 1-hydroxy-3,6,7-trimethoxy-2,8-diprenylxanthone, proxyphylline, pantethine, pangamic acid sodium, zaleplon, sr-2640, rabeprazole sodium, risedronate sodium hydrate, sucralfate sodium (10 mm 10% aq dmso), sucralose, colistin sulfate, nitroglycerin, arsenic trioxide, dipteryxin, benzbromarone, bromperidol, cyproheptadine hydrochloride, clofazimine, benzydamine hydrochloride, doxazosin mesylate, isoetharine mesylate, florfenicol, ethynodiol diacetate, ornidazole, oxantel pamoate, phenformin hydrochloride, protryptyline hydrochloride, nizatidine, nialamide, denatonium benzoate, clemizole hydrochloride, decamethonium bromide, buflomedil hydrochloride, mesalamine, ethamivan, butyl paraben, cefalonium, imipenem, lactobionic acid, methionine sulfoximine (l), metyrapone, mevalonic acid lactone, naftopidil, oxalamine citrate, picotamide, repaglinide, risperidone, eticlopride hydrochloride, sotalol hydrochloride, thioguanosine, tiratricol, meta-cresyl acetate, trolox, tyloxapol, chloropyramine hydrochloride, clorgiline hydrochloride, debrisoquin sulfate, enilconazole, finasteride, idazoxan hydrochloride, kawain, meticrane, moroxydine hydrochloride, moxisylyte hydrochoride, pentetic acid, proscillaridin, oxedrine, sulfadoxine, trimetazidine dihydrochloride, menaquinone-4, pentagastrin, protirelin, allylisothiocyanate, acesulfame potassium, afalanine, sulbentine, sulfacarbamide, tenylidone, modaline sulfate, adrenolone hydrochloride, allylthiourea, dexfosfoserine, dimetridazole, carglumic acid, dimethyl fumarate, benzoclidine, palmidrol, nikethamide, meparfylon, fomepizole, felbinac, hexylene glycol, fluroxene, selenomethionine, idramantone, guaiacol, piconol, picolamine, oxiniacic acid, paroxypropione, ethanolamine oleate, phenothiazine, nonivamide, nitroxoline, hymechrome, procodazole, pimagedine hydrochloride, isovaleramide, levcycloserine, casanthranol [cascaroside a shown], eprodisate disodium, imexon, nitarsone, meglutol, docusate sodium, pidolic acid, cinoctramide, octocrylene, terpene hydrate, symclosene, tiopronin, tolonium chloride, sodium monofluorophosphate, troclosene potassium, nicopholine, iproheptine, isobutamben, membutone, mexeneone, thymopentin, filipin, ammonium lactate, 2,6-dihydroxy-4-methoxytoluene, retusin, creatinine, tetrahydrosappanone a trimethyl ether, catechin pentaacetate, butylated hydroxytoluene, 4-hydroxy-6-methylpyran-2-one, 3-methoxycatechol, n-methylanthranilic acid, mevastatin, diethyltoluamide, paeonol, epicatechin 3,5-digallate, mundulone acetate, androsta-1,4-dien-3,17-dione, cortisone, hydroxyprogesterone, menthyl benzoate, limonin, tilorone, clomipramine hydrochloride, chlormezanone, astemizole, hydroxytacrine maleate, anhydroglucose, tacrine hydrochloride, 5-methylhydantoin, n-hydroxymethylnicotinamide, acecainide hydrochloride, amoxapine, amiodarone hydrochloride, alprenolol, paraxanthine, buspirone hydrochloride, phenoxybenzamine hydrochloride, 8-cyclopentyltheophylline, levodopa, diazoxide, domperidone, diltiazem hydrochloride, efaroxan hydrochloride, edrophonium chloride, n-methyl (−)ephedrine [1r,2s], kainic acid, glyburide, isradipine, loperamide hydrochloride, loxapine succinate, 1r,2s-phenylpropylamine, nicotine bitartrate, lorglumide sodium, pinacidil, mianserin hydrochloride, mexamine, verapamil hydrochloride, vesamicol hydrochloride, metolazone, podofilox, nipecotic acid, pentylenetetrazol, 1-(2-methoxyphenyl)pierazine hydrochloride, 7-hydroxyethyltheophylline, inosine, biochanin a, and cinnamic acid.

Animals.

Five to twelve week old male C57BL/6J, DBA/2J, and BALB/cJ mice were obtained from The Jackson Laboratory (Bar Harbor, Me.). AHR-deficient mice on a C57BL/6J background (AHR^{-/-}) were bred and maintained under specific pathogen-free conditions[54]. This study was carried out in strict accordance with the recommendations in the Guide for the Care and Use of Laboratory Animals of the National Institutes of Health. All animal experiments were carried out according to institutional guidelines with appropriate IRB approval from the University of Wisconsin-Madison Animal Care and Use Committee, under protocol number M02293-0-09-11.

Cell Culture.

All cell lines and isolated cell preps were cultured in Dulbecco's modified medium with high glucose supplemented with 10% (v/v) fetal bovine serum, 1 mM sodium pyruvate, 2 mM L-glutamine, 10 μM HEPES buffer solution, 0.1 mM minimal essential medium nonessential amino acids, and penicillin-streptomycin at 100 U/ml and 100 μg/mL, respectively (all media reagents from Invitrogen Corp., Carlsbad, Calif.). Cell cultures were maintained in a standard 5% CO2, 37° C. environment. The 101L cell line harbors a stably transfected luciferase reporter driven by 3 upstream DREs[13]. The mouse hepatoma cell line C4 lacks expression of ARNT while the C35 cell line expresses a mutant AHR incapable of nuclear translocation and binding DRE[18,19,55,56]. The C4 and C35 cell lines were provided by Dr. Oliver Hankinson (Los Angeles, Calif.)[18,19] and the 101L cell line was a gift of Dr. Robert Tukey (San Diego, Calif.)[13].

Small Molecule Screen.

The compounds were dissolved in DMSO to generate 1 mM stocks and were arrayed in 384-well plates. High-throughput compound screening was performed in 384-well plates using the 101L reporter cell line and the Biomek automated liquid-handler (Beckman Coulter, Inc., Fullerton, Calif.). To each well, 100 μL of culture media containing ˜8000 cells and 1 μL test compound were added (1% DMSO, v/v). At ˜24 hours post-treatment, luciferase activity was assayed by first removing 60 μL of culture media from the wells and then adding 30 μL of Bright-Glo™ luciferase reagent to the cells (Promega, Madison, Wis.). Induction of luciferase activity was monitored in the Perkin-Elmer Victor 3-V microplate luminometer (Waltman, Mass.). Basal luciferase signal produced from cells treated with DMSO alone served as controls from which the fold-induction of AHR activity was calculated.

Generation of AHR^{d}-15 Cell Line—

The open reading frame of the AHR^{d} gene (PL2047) was amplified by PCR using oligos OL5127 (5′-GAACCATGAGCAGCGGCGCC-3′) (SEQ ID NO:1) and OL5128 (5′-CCCTACAGGAATCCACCAGGTGTGATATC-3′) (SEQ ID NO:2). The product was ligated into the pTARGET™ mammalian expression vector (Promega, Madison, Wis.). The resulting plasmid was transfected into the AHR-deficient cell line, BP8 [15], using EFFECTENE Transfection Reagent (Qiagen Inc., Valencia, Calif.). Stable integrants were selected with 6 mg/ml of G418 for 2 weeks, beginning at 48-hours post-transfection. Clones derived from single cells were screened for their response to nanomolar doses of dioxin or micromolar doses of BNF, as measured by ethoxyresorufin O-deethylase (EROD) activity. Confirmation of AHR^{d} expression in select cell lines was achieved by Western blot using the BEAR-3 anti-AHR antibody that recognizes the PAS domain[57]. The validated cell line used in these studies was designated line AHR^{d}-15.

Transfection and Luciferase Assays—

To determine ED₅₀ for TCDD or SU5416-induced AHR activity, COS-1 cells[58,59] (Sigma; St Louis, Mo.) were transiently transfected with pTarget-mAHR or pTarget-mAHR-A375V expression plasmid, pGudLuc6.1 luciferase reporter plasmid and TK-renilla luciferase plasmid using Lipofectamine™ 2000 (Invitrogen, Carlsbad Calif.). Six hours after transfection, cells were cultured with DMEM media containing TCDD (1×10⁻⁷-1×10⁻¹³M), SU5416 (3×10⁻⁷-1×10⁻¹²M) or vehicle alone (0.1% DMSO). After 4 hours of treatment, cells were assayed with dual Luciferase® reporter assay system (Promega, Madison, Wis.). The expressed luciferase activity was measured by MicroLumat Plus Luminometer (Berthold Technologies, Hartfordshire, UK). The dose-response curves and ED₅₀ values for TCDD and SU5416 were determined using GraphPad Prism 4 software (GraphPad software Inc., La Jolla, Calif.).

For other luciferase assays, a mouse hepatoma cell line H1L6.1c3, stably carrying a dioxin-responsive element (DRE)-driven firefly luciferase reporter gene [a gift from Dr. Denison, University of California, Davis, Calif.[60] was maintained with 0.3 mg/ml G418 in completed DMEM media. Briefly, 0.6×10⁶ cells were seeded in each well of a six-well plate overnight and were then treated with SU5416 or other ligands at the dose described in the text. Cells were lysed by lysis buffer (Promega, Madison, Wis.), and the luciferase assay was performed by using a BD moonlight 3010 luminometer (BD Biosciences, San Jose, Calif.). The relative light unit is the indicator of luciferase expression level.

Validation of SU5416—

To assess the role of the AHR in SU5416-induced DRE-dependent gene transcription, the C35 AHR mutant cell line was transiently transfected with an expression vector containing the AHR^{b-1} cDNA (PL65), the pCH110 lacZ plasmid, and a vector containing a DRE-luciferase construct (PL265). Control samples were mock transfected with the two reporter plasmids and the empty pSPORT vector (PL22), the parent vector from which PL65 was derived. The cells were seeded into 24-well plates at −60% density and transfected with 67 ng of each plasmid DNA. Following 24 hours, 3 μM SU5416, 3 μM BNF or 0.3% (v/v) DMSO was added. The cells were cultured for an additional 18 hours prior to assessment of luciferase and β-galactoside activity using commercial kits (Promega, Madison, Wis.). The ARNT-deficient cell line, C4, was transfected, treated, and assayed as described above for the C35 cells. However, in place of the AHR-bearing plasmid, these cells were transfected with the human ARNT (PL87), plus the luciferase and lacZ reporter plasmids.

Isolation of Hepatic Microsomal Fraction—

Hepatic microsomes were prepared by homogenizing 0.5 g of liver tissue in 5 mL of MENG buffer (25 mM buffer sodium morpholinopropane sulfonate buffer (pH 7.5) containing 0.025% (w/v) NaN₃, 1 mM EGTA and 10% (v/v) glycerol). The homogenate was subjected to centrifugation at 10,000′ g for 20 minutes, followed by centrifugation of the supernatant for 1 hour at 100,000′ g and 4° C. The pellet was dissolved in 15 mM Tris-HCl buffer containing 250 mM sucrose (pH 8.0) and aliquots were stored at −80° C. Protein concentration was determined using the Bicinchoninic Acid Protein Assay Reagent (Pierce Biotechnology, Rockford, Ill.).

High Throughput Ethoxyresorufin O-Deethylase (EROD) Analysis—

High-throughput analysis of EROD activity was assayed by adapting a protocol that was described elsewhere[61,62]. Briefly, cells were seeded into 96-well plates at −60% density and treated with the test compounds for 36 hours. The cells were washed with PBS and lysed with 30 μL water and a cycle of freeze-thaw. To each well, 150 μL of 50 mM Hepes buffer containing 26.7 μM dicumarol and 13.3 μM ethoxyresorufin were added. The samples were incubated at 30° C. for 20 minutes, and 50 μL of 0.5 mM β-NADPH were added to initiate the reaction. Fluorescence of the resorufin product was detected using the 544 nm excitation and 590 nm emission wavelength filter set.

Dose-Dependent Response to SU5416 in Mice—

Five-week old male C57BL/6J and DBA/2J mice were obtained from Jackson Laboratories (Bar Harbor, Me.) and housed at the University of Wisconsin animal facility. Groups of four mice were orally administered 30, 80, or 120 mg of SU5416 per kg of body weight. Control groups were dosed with BNF at 120 mg/kg or an equivalent volume of corn oil (30 mL/kg). After 48 hours, liver tissue was collected for preparation of microsomes and determination of EROD activity.

Photoaffinity Ligand Binding—

hepatic cytosolic fractions were isolated from the livers of male C57BL/6J mice. Cytosolic fractions were diluted to 1 mg of protein per mL of 25 mM sodium morpholinopropane sulfonate buffer (pH 7.5) that contains 0.025% (w/v) NaN₃, 1 mM EGTA, 10% (v/v) glycerol, 15 mM NaCl, 1.0 mM dithiothreitol and 0.10% (v/v) Nonidet NP-40. A 1 nM concentration of radioligand, [¹²⁵I]2-azido-3-iodo-7,8-dibromodibenzo-p-dioxin (¹²⁵Ibr₂N₃DpD), was incubated with increasing concentrations of the competing compound[63]. The binding reactions were performed at 20° C. for 30 min, followed by incubation at 0° C. for 5 min to minimize ligand dissociation. Charcoal and gelatin (respective concentration of 1% and 0.1%, w/v) were added for 10 min at 0° C. to absorb the unbound ligand and then removed by centrifugation. The ¹²⁵Ibr₂N₃DpD-bound cytosolic fractions were UV-irradiated with four Photodyne 300 nm wavelength lamps at a distance of 4 cm for 1 minute. The protein was precipitated by an overnight incubation in acetone at −20° C., collected by centrifugation and washed with cold acetone:water (9:1, v/v). The washed pellet was dissolved in sodium dodecyl sulfate sample buffer and resolved by electrophoresis on a 7.5% (w/v) polyacrylamide gel. Following staining and drying, the gel was exposed to film overnight at −80° C. with an intensifying screen. The 95-kDa AHR^{b-1} band was excised and the amount of ¹²⁵Ibr₂N₃DpD covalently bound was quantified in a γ counter.

Assessment of Ductus Venosus Status—

Timed mating of female AHR^{fxneo/+} mice to male AHR^{fxneo/fxneo} mice was performed[5]. At gestation day E18.5, the pregnant dams were injected i.p. with 110 mg/kg of SU5416 or the vehicle, corn oil. When the pups were 4 weeks of age, the status of the DV was determined by hepatic perfusion with Trypan Blue as previously described[5]. Briefly, each mouse was anesthetized and its liver was flushed with PBS through the cannulated portal vein. The inferior vena cava was incised to allow outflow. Trypan blue was injected through the portal vein until the liver visibly turned blue (in the case of a closed DV) or until the dye was seen exiting the IVC without perfusing the liver (when the DV is open).

Real-Time Quantitative PCR (qPCR)—

Spleen was harvested from mice at the time of euthanasia and single cell suspensions were made. RBCs were eliminated from spleen preps using RBC Lysing Solution (eBioscience). Total RNA was extracted using the reagents: Rneasy Mini Kit and Rnase-Free Dnase Set (Qiagen, Valencia, Calif.). A total of 500 ng total RNA in each group was used for RT reaction (iScript cDNA Synthesis Kit, Bio-Rad, Hercules, Calif.; or High-capacity cDNA Reverse Transcription Kits, Applied Biosystems, Foster City, Calif.). The relative quantitation PCR for IDO1 (Mm00492586-ml) and GAPDH (4352339-E0806018) were performed in the Applied Biosystems 7900HT Fast Real-Time PCR System (Applied Biosystems), and TaqMan Universal PCR Master Mix (Applied Biosystems) was used as a reaction reagent. The relative quantitation PCR for Foxp3, CYP1A1, CYP1B1, and GAPDH were processed by the Bio-Rad iCycler (Bio-Rad) and iQ SYBR Green Supermix (Applied Biosystems) was used as the reaction reagent.

Isolation of Naive CD4⁺ T Cells and T Cell Differentiation—

Naive CD4⁺ T cells were isolated using the CD4⁺ CD62L Isolation Kit (Milenyi Biotec, Auburn, Calif.) and an autoMACS. This kit includes a depletion mixture, including the addition of a CD25 and an anti-TCRγ/δ⁺ Ab. Cells were tested for purity post-sorting and consistently showed >90% purity for CD4⁺ CD62⁺ CD25⁻ cells. Viability at the beginning of culture was typically >98% as seen by trypan blue staining. For quantitative PCR (qPCR) analysis, 2-5×10⁵ cells were cultured in each well of a 96-well round-bottom plate coated with 0.5 μg/ml anti-CD3 and anti-CD28 overnight and then washed with PBS twice before seeding the cells. The naive T cells were maintained in F10 media supplemented with 10% heat-inactivated FBS, 100 μg/ml streptomycin, 100 U/ml penicillin, 50 μm 2-ME, 25 mM HEPES, and 2 mM L-glutamine. Cells in Treg conditions included the addition of TGF-β (5 ng/ml). Th17 conditions includes TGF-β and IL-6.

pDC/T Cell Co-Culture—

Naive CD4⁺ CD25⁻ T cells were isolated from WT and AHR-null mice and co-cultured with BALB/cJ pDCs isolated using the Miltenyi Mouse pDC Isolation Kit (Miltenyi Biotec) at a ratio of 20:1 or 10:1 as previously described (13). SU5416, TCDD (10 nM), or FICZ (100 nM) were added at the start of culture. On day 5, cells were harvested and subjected to qPCR analysis.

Flow Cytometry—

To stain for FoxP3, T cells were first surface stained with anti-CD4 and antiCD25 and then fixed and permeabilized with the Fixation/Permeabilization buffer (ebioscience, San Diego, Calif.) for 30 min at 4° C. Following this, cells were stained with Pacific Blue-conjugated anti-FoxP3. For intracellular IL-17 staining, T cells were first stimulated with 50 ng/ml PMA (Sigma-Aldrich) and 800 ng/ml ionomycin (Sigma-Aldrich) for 4 h in the presence of GolgiStop (BD Pharmingen, San Diego, Calif.) for the final 2 h. Cells were then fixed and permeabilized with the Fixation/Permeabilization buffer (eBioscience) and then stained with PE-conjugated anti-IL-17. All abs were from eBioscience. Flow cytometric analysis was performed using an LSR-II (BD Biosciences). CD39 antibodies for flow cytometry were purchased from Santa Cruz Biotechnology.

### REFERENCES

1. Amacher D E (2010) The effects of cytochrome P450 induction by xenobiotics on endobiotic metabolism in pre-clinical safety studies. Toxicol Mech Methods 20: 159-166.
2. Knerr S, Schrenk D (2006) Carcinogenicity of 2,3,7,8-tetrachlorodibenzo-p-dioxin in experimental models. Mol Nutr Food Res 50: 897-907.
3. Lahvis G P, Lindell S L, Thomas R S, McCuskey R S, Murphy C, et al. (2000) Portosystemic shunting and persistent fetal vascular structures in aryl hydrocarbon receptor-deficient mice. Proc Natl Acad Sci USA 97: 10442-10447.
4. Thackaberry E A, Gabaldon D M, Walker M K, Smith S M (2002) Aryl hydrocarbon receptor null mice develop cardiac hypertrophy and increased hypoxia-inducible factor-lalpha in the absence of cardiac hypoxia. Cardiovasc Toxicol 2: 263-274.
5. Walisser J A, Bunger M K, Glover E, Bradfield C A (2004) Gestational exposure of Ahr and Arnt hypomorphs to dioxin rescues vascular development. Proc Natl Acad Sci USA 101: 16677-16682.
6. Quintana F J, Basso A S, Iglesias A H, Korn T, Farez M F, et al. (2008) Control of T(reg) and T(H)17 cell differentiation by the aryl hydrocarbon receptor. Nature 453: 65-71.
7. Veldhoen M, Hirota K, Westendorf A M, Buer J, Dumoutier L, et al. (2008) The aryl hydrocarbon receptor links T(H)17-cell-mediated autoimmunity to environmental toxins. Nature 453: 106-109.
8. Martin B, Hirota K, Cua D J, Stockinger B, Veldhoen M (2009) Interleukin-17-producing gammadelta T cells selectively expand in response to pathogen products and environmental signals. Immunity 31: 321-330.
9. Kiss E A, Vonarbourg C, Kopfmann S, Hobeika E, Finke D, et al. (2011) Natural aryl hydrocarbon receptor ligands control organogenesis of intestinal lymphoid follicles. Science 334: 1561-1565.
10. Lee J S, Cella M, McDonald K G, Garlanda C, Kennedy G D, et al. (2012) AHR drives the development of gut ILC22 cells and postnatal lymphoid tissues via pathways dependent on and independent of Notch. Nat Immunol 13: 144-151.
11. Qiu J, Heller J J, Guo X, Chen Z M, Fish K, et al. (2012) The aryl hydrocarbon receptor regulates gut immunity through modulation of innate lymphoid cells. Immunity 36: 92-104.
12. McMillan B J, Bradfield C A (2007) The aryl hydrocarbon receptor sans xenobiotics: endogenous function in genetic model systems. Mol Pharmacol 72: 487-498.
13. Postlind H, Vu T P, Tukey R H, Quattrochi L C (1993) Response of human CYP1-luciferase plasmids to 2,3,7,8-tetrachlorodibenzo-p-dioxin and polycyclic aromatic hydrocarbons. Toxicol Appl Pharmacol 118: 255-262.
14. Swanson H I, Bradfield C A (1993) The AH-receptor: genetics, structure and function. Pharmacogenetics 3: 213-230.
15. Weiss C, Kolluri S K, Kiefer F, Gottlicher M (1996) Complementation of Ah receptor deficiency in hepatoma cells: negative feedback regulation and cell cycle control by the Ah receptor. Exp Cell Res 226: 154-163.
16. Burke M D, Thompson S, Elcombe C R, Halpert J, Haaparanta T, et al. (1985) Ethoxy-, pentoxy- and benzyloxyphenoxazones and homologues: a series of substrates to distinguish between different induced cytochromes P-450. Biochem Pharmacol 34: 3337-3345.
17. Niwa A, Kumaki K, Nebert D W, Poland A P (1975) Genetic expression of aryl hydrocarbon hydroxylase activity in the mouse. Distinction between the “responsive” homozygote and heterozygote at the Ah locus. Arch Biochem Biophys 166: 559-564.
18. Karenlampi S O, Legraverend C, Gudas J M, Carramanzana N, Hankinson O (1988) A third genetic locus affecting the Ah (dioxin) receptor. J Biol Chem 263: 10111-10117.
19. Reisz-Porszasz S, Probst M R, Fukunaga B N, Hankinson O (1994) Identification of functional domains of the aryl hydrocarbon receptor nuclear translocator protein (ARNT). Mol Cell Biol 14: 6075-6086.
20. Hogenesch J B, Chan W K, Jackiw V H, Brown R C, Gu Y Z, et al. (1997) Characterization of a subset of the basic-helix-loop-helix-PAS superfamily that interacts with components of the dioxin signaling pathway. J Biol Chem 272: 8581-8593.
21. Poland A, Glover E, Ebetino F H, Kende A S (1986) Photoaffinity labeling of the Ah receptor. J Biol Chem 261: 6352-6365.
22. Walisser J A, Bunger M K, Glover E, Harstad E B, Bradfield C A (2004) Patent ductus venosus and dioxin resistance in mice harboring a hypomorphic Arnt allele. J Biol Chem 279: 16326-16331.
23. Mendel D B, Schreck R E, West D C, Li G, Strawn L M, et al. (2000) The angiogenesis inhibitor SU5416 has long-lasting effects on vascular endothelial growth factor receptor phosphorylation and function. Clin Cancer Res 6: 4848-4858.
24. Munn D H, Mellor A L, Rossi M, Young J W (2005) Dendritic cells have the option to express IDO-mediated suppression or not. Blood 105: 2618.
25. Mezrich J D, Fechner J H, Zhang X, Johnson B P, Burlingham W J, et al. (2010) An interaction between kynurenine and the aryl hydrocarbon receptor can generate regulatory T cells. J Immunol 185: 3190-3198.
26. Vogel C F, Goth S R, Dong B, Pessah I N, Matsumura F (2008) Aryl hydrocarbon receptor signaling mediates expression of indoleamine 2,3-dioxygenase. Biochem Biophys Res Commun 375: 331-335.
27. Chen W, Liang X, Peterson A J, Munn D H, Blazar B R (2008) The indoleamine 2,3-dioxygenase pathway is essential for human plasmacytoid dendritic cell-induced adaptive T regulatory cell generation. J Immunol 181: 5396-5404.
28. Gandhi R, Kumar D, Burns E J, Nadeau M, Dake B, et al. (2010) Activation of the aryl hydrocarbon receptor induces human type 1 regulatory T cell-like and Foxp3(+) regulatory T cells. Nat Immunol 11: 846-853.
29. Fong T A, Shawver L K, Sun L, Tang C, App H, et al. (1999) SU5416 is a potent and selective inhibitor of the vascular endothelial growth factor receptor (Flk-1/KDR) that inhibits tyrosine kinase catalysis, tumor vascularization, and growth of multiple tumor types. Cancer Res 59: 99-106.
30. Kieran M W, Supko J G, Wallace D, Fruscio R, Poussaint T Y, et al. (2009) Phase I study of SU5416, a small molecule inhibitor of the vascular endothelial growth factor receptor (VEGFR) in refractory pediatric central nervous system tumors. Pediatr Blood Cancer 52: 169-176.
31. Overmoyer B, Fu P, Hoppel C, Radivoyevitch T, Shenk R, et al. (2007) Inflammatory breast cancer as a model disease to study tumor angiogenesis: results of a phase IB trial of combination SU5416 and doxorubicin. Clin Cancer Res 13: 5862-5868.
32. Fury M G, Zahalsky A, Wong R, Venkatraman E, Lis E, et al. (2007) A Phase II study of SU5416 in patients with advanced or recurrent head and neck cancers. Invest New Drugs 25: 165-172.
33. Kim Y S, Hong S W, Choi J P, Shin T S, Moon H G, et al. (2009) Vascular endothelial growth factor is a key mediator in the development of T cell priming and its polarization to type 1 and type 17 T helper cells in the airways. J Immunol 183: 5113-5120.
34. Roscoe W A, Welsh M E, Carter D E, Karlik S J (2009) VEGF and angiogenesis in acute and chronic MOG((35-55)) peptide induced EAE. J Neuroimmunol 209: 6-15.
35. Edelbauer M, Datta D, Vos I H, Basu A, Stack M P, et al. (2010) Effect of vascular endothelial growth factor and its receptor KDR on the transendothelial migration and local trafficking of human T cells in vitro and in vivo. Blood 116: 1980-1989.
36. Flegal K M (2005) Epidemiologic aspects of overweight and obesity in the United States. Physiol Behav 86: 599-602.
37. Schmidt J V, Carver L A, Bradfield C A (1993) Molecular characterization of the murine Ahr gene. Organization, promoter analysis, and chromosomal assignment. J Biol Chem 268: 22203-22209.
38. Poland A, Glover E, Taylor B A (1987) The murine Ah locus: a new allele and mapping to chromosome 12. Mol Pharmacol 32: 471-478.
39. Okey A B, Vella L M, Harper P A (1989) Detection and characterization of a low affinity form of cytosolic Ah receptor in livers of mice nonresponsive to induction of cytochrome P1-450 by 3-methylcholanthrene. Mol Pharmacol 35: 823-830.
40. Harper P A, Golas C L, Okey A B (1991) Ah receptor in mice genetically “nonresponsive” for cytochrome P4501A1 induction: cytosolic Ah receptor, transformation to the nuclear binding state, and induction of aryl hydrocarbon hydroxylase by halogenated and nonhalogenated aromatic hydrocarbons in embryonic tissues and cells. Mol Pharmacol 40: 818-826.
41. Flaveny C A, Murray I A, Chiaro C R, Perdew G H (2009) Ligand selectivity and gene regulation by the human aryl hydrocarbon receptor in transgenic mice. Mol Pharmacol 75: 1412-1420.
42. Kimura A, Naka T, Nohara K, Fujii-Kuriyama Y, Kishimoto T (2008) Aryl hydrocarbon receptor regulates Stat1 activation and participates in the development of Th17 cells. Proc Natl Acad Sci USA 105: 9721-9726.
43. Ishimaru N, Takagi A, Kohashi M, Yamada A, Arakaki R, et al. (2009) Neonatal exposure to low-dose 2,3,7,8-tetrachlorodibenzo-p-dioxin causes autoimmunity due to the disruption of T cell tolerance. J Immunol 182: 6576-6586.
44. Quintana F J, Murugaiyan G, Farez M F, Mitsdoerffer M, Tukpah A M, et al. (2010) An endogenous aryl hydrocarbon receptor ligand acts on dendritic cells and T cells to suppress experimental autoimmune encephalomyelitis. Proc Natl Acad Sci USA 107: 20768-20773.
45. Mendel D B, Laird A D, Smolich B D, Blake R A, Liang C, et al. (2000) Development of SU5416, a selective small molecule inhibitor of VEGF receptor tyrosine kinase activity, as an anti-angiogenesis agent. Anticancer Drug Des 15: 29-41.
46. Cao Y (2009) Tumor angiogenesis and molecular targets for therapy. Front Biosci 14: 3962-3973.
47. Singer K, Gottfried E, Kreutz M, Mackensen A (2011) Suppression of T-cell responses by tumor metabolites. Cancer Immunol Immunother.
48. Charlton M (2004) Nonalcoholic fatty liver disease: a review of current understanding and future impact. Clin Gastroenterol Hepatol 2: 1048-1058.
49. Motzer R J, Hutson T E, Tomczak P, Michaelson M D, Bukowski R M, et al. (2007) Sunitinib versus interferon alfa in metastatic renal-cell carcinoma. N Engl J Med 356: 115-124.
50. Carvalho J F, Blank M, Shoenfeld Y (2007) Vascular endothelial growth factor (VEGF) in autoimmune diseases. J Clin Immunol 27: 246-256.
51. Reinders M E, Sho M, Izawa A, Wang P, Mukhopadhyay D, et al. (2003) Proinflammatory functions of vascular endothelial growth factor in alloimmunity. J Clin Invest 112: 1655-1665.
52. Hauben E, Gregori S, Draghici E, Migliavacca B, Olivieri S, et al. (2008) Activation of the aryl hydrocarbon receptor promotes allograft-specific tolerance through direct and dendritic cell-mediated effects on regulatory T cells. Blood 112: 1214-1222.
53. Hu W, Sorrentino C, Denison M S, Kolaja K, Fielden M R (2007) Induction of cyp1a1 is a nonspecific biomarker of aryl hydrocarbon receptor activation: results of large scale screening of pharmaceuticals and toxicants in vivo and in vitro. Mol Pharmacol 71: 1475-1486.
54. Schmidt J V, Su G H, Reddy J K, Simon M C, Bradfield C A (1996) Characterization of a murine Ahr null allele: involvement of the Ah receptor in hepatic growth and development. Proc Natl Acad Sci USA 93: 6731-6736.
55. Wood S M, Gleadle J M, Pugh C W, Hankinson O, Ratcliffe P J (1996) The role of the aryl hydrocarbon receptor nuclear translocator (ARNT) in hypoxic induction of gene expression. Studies in ARNT-deficient cells. J Biol Chem 271: 15117-15123.
56. Sun W, Zhang J, Hankinson O (1997) A mutation in the aryl hydrocarbon receptor (AHR) in a cultured mammalian cell line identifies a novel region of AHR that affects DNA binding. J Biol Chem 272: 31845-31854.
57. Bunger M K, Moran S M, Glover E, Thomae T L, Lahvis G P, et al. (2003) Resistance to 2,3,7,8-tetrachlorodibenzo-p-dioxin toxicity and abnormal liver development in mice carrying a mutation in the nuclear localization sequence of the aryl hydrocarbon receptor. J Biol Chem 278: 17767-17774.
58. Jensen F C, Girardi A J, Gilden R V, Koprowski H (1964) Infection of Human and Simian Tissue Cultures with Rous Sarcoma Virus. Proc Natl Acad Sci USA 52: 53-59.
59. Gluzman Y (1981) SV40-transformed simian cells support the replication of early SV40 mutants. Cell 23: 175-182.
60. Rushing S R, Denison M S (2002) The silencing mediator of retinoic acid and thyroid hormone receptors can interact with the aryl hydrocarbon (Ah) receptor but fails to repress Ah receptor-dependent gene expression. Arch Biochem Biophys 403: 189-201.
61. Sanderson J T, Aarts J M, Brouwer A, Froese K L, Denison M S, et al. (1996) Comparison of Ah receptor-mediated luciferase and ethoxyresorufin-O-deethylase induction in H4IIE cells: implications for their use as bioanalytical tools for the detection of polyhalogenated aromatic hydrocarbons. Toxicol Appl Pharmacol 137: 316-325.
62. Kennedy S W, Jones S P (1994) Simultaneous measurement of cytochrome P4501A catalytic activity and total protein concentration with a fluorescence plate reader. Anal Biochem 222: 217-223.
63. Bradfield C A, Kende A S, Poland A (1988) Kinetic and equilibrium studies of Ah receptor-ligand binding: use of [125I]2-iodo-7,8-dibromodibenzo-p-dioxin. Mol Pharmacol 34: 229-237.
64. Hoff, P M, Wolff, RA, Bogaard, K, Waldrum, S, Abbruzzese, J L. (2006) A phase study of escalating doses of the tyrosine kinase inhibitor Semaxanib (SU5416) in combination with irinotecan in patients with advanced colorectal carcinoma. Jpn J Clin Oncol. 36 (2) 100-103.

We claim:

1. A method of treating a patient with an autoimmune disease or inflammatory disorder comprising the step of:
(a) treating the patient with an effective amount of SU5416,
wherein at least one symptom of disease is reduced or alleviated.

2. The method of claim 1, wherein the patient is treated when at least one symptom of the disease is diagnosed.

3. The method of claim 2, wherein the symptom is at acute phase.

4. The method of claim 1 further comprising the step of treating the patient with at least one additional therapeutic compound.

5. The method of claim 4, wherein the additional compound enhances T-cell differentiation to regulatory T cells.

6. The method of claim 5, wherein the compound is selected from the group consisting of Thymoglobulin (rATG), Campath, costimulatory blockade, infliximab, etanercept, adalimumab, golimumab, natalizumab, cytokines IL-10, IL-2, TGF-β, NSAIDs, corticosteroids, immune suppressants and TNF-α inhibitors.

7. The method of claim 6, wherein the immune suppressant is selected from the group consisting of calcineurin inhibitors, corticosteroids, anti-proliferatives, and mTOR inhibitors.

8. The method of claim 1, wherein the dose of SU5416 in the composition is 30-150 mg/m².

9. The method of claim 8, wherein the dose of SU5416 in the composition is 85-145 mg/m².

10. The method of claim 1, wherein the acute symptoms are treated for a few weeks to months.

11. The method of claim 1, wherein the disease treated is an autoimmune disease or inflammatory disorder selected from the group consisting of rheumatoid arthritis, psoriatic arthritis diabetes, multiple sclerosis, interstitial fibrosis, lupus, glomerulonephritis, Crohn's Disease, inflammatory bowel disease, psoriasis and autoimmune eye diseases (uveitis).

12. A method of treating a transplant patient comprising the step of
(a) treating a patient with an effective amount of SU5416,
wherein at least one symptom of transplant rejection is reduced or alleviated.

13. The method of claim 12, wherein the patient is treated when at least one symptoms of the transplant rejection is diagnosed.

14. The method of claim 13, wherein the symptom is at acute phase.

15. The method of claim 12 further comprising the step of treating the patient with at least one additional therapeutic compound.

16. The method of claim 15, wherein the additional compound enhances T-cell differentiation to regulatory T cells.

17. The method of claim 16, wherein the additional compound is selected from the group consisting of Thymoglobulin (rATG), Campath, costimulatory blockade, infliximab, etanercept, adalimumab, golimumab, natalizumab, cytokines IL-10, IL-2, TGF-β, NSAIDs, corticosteroids, immune suppressants and TNF-α inhibitors.

18. The method of claim 17, wherein the immune suppressant is selected from the group consisting of calcineurin inhibitors, corticosteroids, anti-proliferatives, and mTOR inhibitors,

19. The method of claim 12, wherein the disease treated is a solid organ transplant.

20. The method of claim 12, wherein the solid organ transplant is selected from the group consisting of lung transplant, bronchiolitis-obliterans syndrome (BOS), heart transplant, kidney transplant, liver transplant, pancreas transplant, and corneal transplant.

21. The method of claim 12, wherein the patient are treated for a few weeks to months.

22. The method of claim 12, wherein the amount of SU5416 in the treatment is 30-150 mg/m².

23. The method of claim 22, wherein the amount of SU5416 in the treatment is 85-145 mg/m².

24. A composition for treating autoimmune disease or transplant rejection comprising
(a) a effective amount of SU5416; and
(b) at least one additional therapeutic compound.

25. The composition of claim 24, wherein the additional therapeutic compound enhances T-cell differentiation to regulatory T cells.

26. The composition of claim 25, wherein the additional compound is selected from the group consisting of Thymoglobulin (rATG), Campath, costimulatory blockade, infliximab, etanercept, adalimumab, golimumab, natalizumab, cytokines IL-10, IL-2, TGF-β, NSAIDs, corticosteroids, immune suppressants and TNF-α inhibitors.

27. The composition of claim 24, wherein the composition treats acute autoimmune disease.

28. The composition of claim 24, wherein the composition treats acute transplant rejection.

29. A method of discovering new therapeutic compounds, comprising the steps of:
(a) examining a target chemical for similar structure or function to SU5416, and
(b) identifying a chemical with sufficient functional similarity to SU5416,
wherein the treatment with the test chemical produces therapeutic results.

30. The method of claim 29 additionally comprising the step of (c) treating an autoimmune or transplant patient with the identified chemical.

31. The method of claim 30, wherein the suitable chemical equivalent of SU5416 would activate AHR.

32. The method of claim 31, wherein the suitable chemical equivalent of SU5416 would inhibit VEGFR.


SEQUENCE LISTING (2 sequences)

SEQ ID NO: 1 - DNA, 20 bp, Artificial Sequence
  Oligos
gaaccatgagcagcggcgcc

SEQ ID NO: 2 - DNA, 29 bp, Artificial Sequence
  Oligos OL5128
ccctacaggaatccaccaggtgtgatatc